(19) United States
(12) Patent Application Publication
(10) Pub. No.: US 2008/0045865 A1
(43) Pub. Date: Feb. 21, 2008

(54) Nanoparticle Mediated Ultrasound Therapy and Diagnostic Imaging

(76) Inventor: Hanoch Kislev, Zichron Yaakov (IL)

Correspondence Address: FENNEMORE CRAIG, 3003 NORTH CENTRAL AVENUE, SUITE 2600, PHOENIX, AZ, 85012, US

(21) Appl. No.: 11/718,778

(22) PCT Filed: Nov. 13, 2005
(86) PCT No.: PCT/IL05/01189

§ 371 Date: Jun. 17, 2007

Related U.S. Application Data
(60) Provisional application No. 60/627,391, filed on Nov. 12, 2004.
(60) Provisional application No. 60/643,291, filed on Jan. 12, 2005.

(51) Int. Cl. A61N 7/02; A61N 5/00

(52) U.S. Cl. 601/3; 607/88; 977/904

(57) ABSTRACT

The present invention relates to systems and methods for localized delivery of heat, useful for localized imaging and treatment of a biological material. The systems and methods of the invention can be utilized for localized treatment of cancer, inflammation or other disorders involving over proliferation of tissue, and for tissue repair. The method comprises exposing nanoparticles to electromagnetic radiation under conditions wherein the nanoparticles generate microbubbles which emit heat when exposed to ultrasonic radiation.

### FIELD OF THE INVENTION

The present invention relates to a system and method for localized delivery of heat, useful for localized imaging and treatment of a biological material, particularly for tissue repair and localized treatment of cancer, inflammation or other disorders involving over proliferation of tissue. The methods comprise exposing nanoparticles to electromagnetic radiation under conditions wherein the nanoparticles generate microbubbles which emit and propagate heat when further exposed to ultrasonic radiation.

### BACKGROUND OF THE INVENTION

Localized heating of cells and tissues is desirable in many applications. Precise, localized heating has been shown to have therapeutic benefits, while minimizing the collateral damage to nearby cells and tissue. The therapeutic effects of thermal ablation range from the destruction of cancerous cells and tumors, to the therapeutic or cosmetic removal of benign tumors and other undesirable tissues.

In addition to their minimally invasive nature, thermal therapeutic procedures are relatively simple to perform and therefore have the potential of improving recovery time, reducing complication rates and hospital stay. Thermal delivery methods suitable for local tissue ablation include focused ultrasound, laser induced thermal therapy, microwave and Radio Frequency (RF) ablation, and nanoparticle-based thermal ablation.

Ultrasound has successfully provided a means by which thermal therapies can be performed extracorporally. Therapeutic applications of ultrasound may be divided into two major categories: applications that employ low intensity (0.125-3 W/cm²) and those that employ higher intensities (>5 W/cm²). Ultrasound radiation in a frequency range of about 1-3 MHz can penetrate deep into the human body.

The attenuation coefficient α for many types of human tissue to ultrasound radiation is expressed as —

$$\frac{α}{f} = 1 * {10}^{- 7} {cm}^{- 1} sec \qquad ( 1 )$$

wherein f denotes frequency in MHz.

[Christensen D. A. in “Ultrasonic Bioinstruments,” Chapter 4, Wiley John & Sons (1988)]. For example, the attenuation of ultrasound beam of 2 MHz, passing through 5 cm of average human tissue would be approximately 65%. This property enables deep penetration of ultrasound into human tissue and opens possibilities to induce hyperthermia of cells or tissues deep in the body.

Low intensity ultrasound is commonly used to stimulate normal physiological responses to injury, for example in physiotherapy, or to accelerate processes, such as the transport of drugs across the skin. In such applications efforts are made to minimize collateral tissue damage, including minimizing excessive tissue heating, typically by reducing the treatment time and/or delivering the ultrasound in a pulsed manner. Low power density ultrasound is not useful for treating cells or tissues by hyperthermia due to only a slight difference between the absorption rate of a diseased and a healthy tissue and the acoustic properties variations in the human body, which may cause damage to adjacent healthy cells or tissues. It is possible to use low power ultrasound and to administer the targeted tissue with contrast agents characterized by a high cross section to ultrasound radiation, which can contribute to the localization of the ultrasound radiation deposition. However, the combined use of ultrasound and targeted contrast agents has a basic limitation: it is very hard to accumulate the necessary concentration of contrast agent within the targeted tissue volume. An attempt to load the desired contrast agent concentration often results in forming a microbubble layer around the targeted tissue, which may block the ultrasound radiation from penetrating into the targeted tissue volume.

Applications involving the use of high intensity ultrasound are typically aimed at selectively destroying a tissue by direct hyperthermic processes. High intensity ultrasound-mediated tissue ablation may be categorized according to the way in which the ultrasound energy is delivered to the tissue. Ultrasound may be delivered directly from a transducer to the area to be treated; alternatively, a coupling device, which focuses the ultrasound, may mediate delivery. When provided through a coupling device, ultrasound passing through intervening tissues is usually of low intensity and therefore, relatively non-destructive. However, at the focal point, the accumulated energy is raised to a pre-determined higher intensity and tissue destruction occurs at, or around, the focal point.

In general, therapeutic applications which rely on the use of high intensity focused ultrasound, or “HIFU,” exploit the heat which is generated at the focal point and a number of methods together with devices for achieving focus and tissue ablation have been disclosed (see, e.g., U.S. Pat. Nos. 4,888,746; 5,895,356; 5,938,608 and International Patent Applications WO 97/35518 and WO 99/22652).

However, in many cases the focused ultrasound energy generates a dense microbubble cloud between the targeted and healthy tissue, which blocks the ultrasound radiation. In addition, dense microbubble cloud interaction with HIFU involves the potential occurrence of cavitation events, which, in turn, leads to the formation of destructive, or possibly mutagenic, free radicals [Miller et al., Ultrasound in Med. & Biol. 22:1131(1996)]. Furthermore, the complex nature of the human organs complicates the aiming procedure when using HIFU, and requires the use of guided treatment, typically using on-line magnetic resonance imaging (MRI) instrumentation. The volume and speed of HIFU treatments is limited by the potential destruction of normal tissue within the near field between the target and the ultrasound probe and due to targeting errors.

Nanoparticles offer new capabilities in localized treatment and diagnosis of cells and tissues. Their size enable them to freely circulate through the blood system, penetrate through the uncontrolled blood cells of tumors and diffuse through the interstitial volumes of the targeted tissue. Conjugating targeting materials to nanoparticles increase their tendency to stick onto targeted cells or tissue or even non-cell non-tissue materials like kidney stones. Sufficiently small nanoparticles can penetrate through the cells membrane. These capabilities enable volumetric treatment of the targeted tissue.

Due to the ability to pre-fabricate nanoparticles in virtually any desired shape and composition, they can be optimized to absorb energy through several coupling mechanisms. Currently, these mechanisms include enhanced absorption of optical radiation, coupling of magnetic fields, and to a lesser extent, ultrasound radiation.

U.S. Pat. No. 6,530,944 to West et al. have describes use of specially shaped nanoparticles whose typical size is 100 to 200 nm for photothermial therapy. These nanoparticles (called nanoshells) include a thin metal shell with geometry optimized for maximum absorption of electromagnetic radiation at a prescribed wavelength. For example, the peak interaction cross section of an optimized nanoshell is about four times the cross section of a similar size all-gold nanoparticle. U.S. Pat. No. 6,685,730 discloses the use of these particles for generation of heat which effects the joining of a tissue or materials.

Hirsch et al. [Hirsch L. R. et al., PNAS 100(23):13549-13554, (2003)] described the use of nanoshells with specific peak absorption at the near infrared (NIR) for treating tumors in mice. They injected an appropriate amount of nanoshells to the vicinity of the tumor, and the injected nanoshells were shown to attach preferably to the cancerous cells. An intense NIR light beam was then applied externally in the direction of the tumor loaded with nanoshells. The nanoshells absorbed the beam radiation and converted the power into thermal energy, which in turn was absorbed in the targeted tissue. After a few minutes of exposure to NIR beam, the tissue temperature increased by more than 15° C. to a depth of a few mm.

However, the use of NIR absorbing nanoparticles for photothermal therapy suffers from a basic drawback: the vast nanoparticle volume concentration required for localized heating. For example, doubling the NIR absorption rate in a typical internal tissue whose NIR absorption and scattering cross section are 0.6 cm⁻¹ and 80 cm⁻¹ respectively, would require concentration of 2*10⁹ nanoshells/cm³. The extremely slow diffusion rate of NIR optimized nanoshells (with a diameter of about 200 nm) makes it very hard to accumulate such concentrations within the volume of a tumor whose size is larger than one cm.

Another unsolved problem of photothermal treatment is the total penetration depth of electromagnetic radiation including NIR, in a tissue. Even for optimized NIR wavelengths, increasing the temperature of a small tumor by 15° C., at a depth larger than a few mm, would require prolonged application of NIR power densities of 5-10 W/cm². The American National Standards Institute (ANSI) regulations [ANSI Regulations Z136.1 (1993)] limit prolonged human skin exposure to infrared light flux to below 1 W/cm². Exposure above threshold may induce deep burns, partially due to sub-dermal back scattering which increases the local internal tissue damage near the light source.

There are several ways to overcome this limitation including the use of smaller NIR absorbing nanoparticles and reducing the required temperature by combining the photothermal treatment with other treatment modalities. Chen et al., [Chen J. et al., Nanoletters, 5(3):473-477, (2005)] suggested another type of NIR absorbing nanoparticles called nanocages mainly as optical coherence tomography (OCT) contrast agents. The size of these nanocages is 40 nm and thus they can penetrate much faster through the poorly defined blood vessels of a developed tumor. However, the required nanocage concentration required for doubling the effective absorption of typical internal tissue would be very high, in the range of 2*10¹⁰ nanocage/cm³.

Ultrasound could be the optimal energy delivery tool for hyperthermia in combination with suitable targeted nanoparticles. Its relative low cost per Watt and the significant penetration depth into human tissue indicates it as an energy source for such application. Unfortunately, the size of nanoparticles which can penetrate through malignant blood vessels (40-200 nm) relates to a very small interaction cross section at the linear range of interaction with ultrasound radiation.

Larina et al [Larina I. V. et al., Technol Cancer Res. Treat. 4:217-226, (2005)] suggested a method for coupling ultrasound with nanoparticles. They irradiated mice inoculated with KM20 glioma tumor with high power 20 kHz ultrasound radiation in combination with intravenous injection of 100 and 280 nm polystyrene nanoparticles. They found that the absorbed ultrasound energy combined with chemotherapy could kill glioma tissue effectively. They also described enhanced chemotherapy diffusion in the interstitium due to dynamic pulsation of the ultrasound-irradiated microbubbles. However, the required nanoparticle concentration was found to be 1*10¹¹ particles/cm³, which concentration is extremely hard to achieve in the tumor volume.

Diagnostic imaging is an important tool for identification and three-dimensional location of diseased tissue and cells. Diagnostic imaging can also indicate the location and boundaries of viable diseased cell or tissue during and after certain treatment, in particular minimally invasive procedures. The typical diagnostic imaging methods used are ultrasound, MRI and X-ray. Ultrasound is an important diagnostic imaging technique which, unlike X-rays, does not expose the patient to the harmful effects of ionizing radiation. Moreover, unlike magnetic resonance imaging, ultrasound is relatively inexpensive and can be conducted as a portable examination. The imaging principle is based on partial reflections from interfaces between tissues and fluids. Unfortunately, there are minor differences between the acoustic impedance difference of healthy and diseased tissue.

Ultrasound diagnostic imaging of diseased tissues is nowadays performed after administering contrast agents to the patient. When ultrasound waves encounter low-density high elasticity interfaces (like contrast agents), the changes in acoustic impedance result in a more intense reflection of sound waves and a more intense signal in the ultrasound image. The contrast agent particle size is a few microns and they are typically coated with attachment promoters which enhance their tendency to attach to the targeted tissue.

Diagnostic imaging is conducted by attachment of ultrasound probe to a free patient surface and transmitting low power ultrasound towards the suspected tissue direction. Typical average ultrasound power range of 1 to 125 mW/cm² and the typical frequency is between 1 and 3 MHz for contrast agents diagnostic imaging. The operation mode may be continuous or composed of certain sequences of pulse train. The reflected echo is received in the probe and converted into an electrical signal which in turn is converted by a suitable CPU into an image of the tissue where the enhanced regions are volumes filled with contrast agent.

Due to their size (a few microns), typical contrast agents do not accumulate in the diseased small vessels and do not penetrate into the interstitial volume. Therefore, the ultrasound images tend to show the main blood vessels of the diseased tissue and not its borders or extent. The tendency of nanoparticles to attach to the targeted tissue may be utilized for tissue imaging if they could generate microbubbles. Unfortunately, nanoparticles whose size is smaller than 100 nm may carry negligible gas content which is hardly practical for imaging purposes. Thus, it is highly desirable to have nanoparticles that generate microbubbles to utilize their advantages for whole diseased tissue imaging.

In view of the foregoing, there is a recognized need for, and it would be highly advantageous to have, systems and methods for coupling ultrasound radiation energy with nanoparticles to induce enhanced, localized, targeted hyperthermia in a cell or a tissue, for therapeutic, diagnostic and imaging purposes.

### SUMMARY OF THE INVENTION

The present invention provides systems and methods for use in cell and tissue therapy and imaging. The primary object of the present invention is to provide a system and method for inducing enhanced, localized, targeted hyperthermia in such cell and tissue, while minimizing collateral damage to surrounding normal cells and tissue, as well as providing a means for precise imaging of the diseased tissue borders and volume.

The present invention discloses that, unexpectedly, there are parameters for regimes where the vapor microbubbles generated by heated nanoparticle clusters or agglomerates can be stabilized and propagated by low power ultrasound radiation. The present invention further discloses that the stabilized microbubbles dramatically increase the local absorption of ultrasound radiation. The absorbed ultrasound radiation is converted to heat which is emitted to the microbubble environment. The present invention also discloses that the large cross section of the stabilized microbubbles result in a significantly lower concentration of nanoparticles required for emitting heat of a certain degree, in comparison to previously known methods.

According to the present invention, nanoparticles with enhanced absorption of electromagnetic radiation can be administered to cells or tissue, exposed to electromagnetic radiation and in turn induce microbubbles. These cells or tissue are then exposed to ultrasound radiation, which is efficiently absorbed by the microbubbles while emitting heat to their surrounding.

The teachings of the present invention are advantageous over previously known methods for localized delivery of heat into cells or tissue as it requires lower concentrations of nanoparticles at the targeted area as well as lower average intensity of ultrasound power density.

According to one aspect, the present invention provides a system for localized delivery of heat to a cell or a tissue preloaded with nanoparticles comprising:
(a) an electromagnetic radiation source configured to irradiate the nanoparticles to induce the production of microbubbles by said nanoparticles;
(b) a therapeutic ultrasonic wave generating source configured to irradiate the microbubbles as to induce heat production by said microbubbles; and
(c) driving means coupled to the therapeutic ultrasonic wave generating source for driving said therapeutic ultrasonic source with a drive signal to generate therapeutic ultrasonic waves.

The electromagnetic radiation source can be selected from a group consisting of, but not limited to, a plurality of light emitting diode (LED) lamp, gaseous flash lamp, diode laser pumped flash lamp or solid state laser, diode laser, or a gaseous laser.

The electromagnetic radiation can be delivered to the nanoparticles preloaded to the cell or tissue by various methods as known to a person skilled in the art. According to certain embodiments, the system further comprises a light guide to target the electromagnetic radiation from the electromagnetic source to the cell or tissue. According to one embodiment, coupling the electromagnetic radiation from the light source to the light guide is attained by means of one or more suitable lenses, lens array, one or more concentrating mirrors, or a combination thereof.

According to certain embodiments, the electromagnetic radiation used to irradiate the nanoparticles is selected from the group consisting of ultraviolet, visible and infrared radiation. According to one currently preferred embodiment, the electromagnetic radiation is infrared radiation in the ranges of from about 800 to about 1300 nm. The electromagnetic operation mode may be repetitive pulse or any other time sequence suitable for generation of microbubbles from appropriate nanoparticles. According to one currently preferred embodiment, the electromagnetic source operation mode is a pulsed mode with pulse width which ranges from 0.01 to 10 microseconds.

The electromagnetic radiation source can be selected from a group consisting of, but not limited to, a plurality of light emitting diode (LED) lamp, gaseous flash lamp, diode laser pumped flash lamp or solid state laser, diode laser, or a gaseous laser. The ultrasound source could be continuous, modulated, coupled to slightly focusing apparatus, and include one or more ceramic transducers, or other suitable ultrasound generating transducers.

According to other embodiments, the source of therapeutic ultrasonic waves (also defined herein as “the therapeutic ultrasonic source”) comprises a housing, the housing comprising at least one piezoelectric transducer element. According to some embodiments, the piezoelectric transducer element is made of a material selected from the group consisting of quartz, barium titanate, lead zirconium titanate and poly(vinylidene fluoride).

According to additional embodiments, the driving means comprises radio-frequency (RF) signal generator and an amplifier that amplifies the RF signal pulses to produce drive signal. According to one embodiment, the driving means is coupled to the therapeutic ultrasonic source through an electric cable, such that the drive signal is applied to the piezoelectric transducer elements of said therapeutic ultrasonic source through the electric cable. Typically, the driving means is located apart from the therapeutic ultrasonic source to avoid exchange of excess vibrations and excess heat. According to certain embodiments, the therapeutic ultrasonic source generates ultrasound radiation at a frequency range between 0.5 and 7.5 MHz. Ultrasound is preferably applied at peak power levels of from about 0.05 W/cm² to about 20 W/cm². The present invention now discloses that providing low intensity ultrasound radiation, at an average power level of from 01.25 to 3 W/cm², is sufficient for significant heat generation by the microbubbles produced according to the teaching of the invention. The Ultrasound radiation can be applied as a continuous wave ultrasound or as a pulsed wave. The ultrasound radiation pulse width preferably ranges between 1 microsecond and 0.5 second.

According to certain embodiments, the system further comprises a focusing device coupled to the therapeutic ultrasonic source.

According to other embodiments, the preloaded nanoparticles are present at a concentration in the range of 10⁵ to 10⁹ nanoparticles/cm³, preferably in the range of 3*10⁵ to 3*10⁷ nanoparticles/cm³. According to additional embodiments, the nanoparticles have an enhanced photothermal cross-section for the electromagnetic source, i.e., the photothermal cross-section is enhanced to at least the physical cross section of the nanoparticle.

According to another aspect, the present invention provides a method for inducing localized delivery of heat to a cell or a tissue comprising:
(a) administering nanoparticles to the cell or tissue;
(b) irradiating the nanoparticles administered to said cell or tissue by electromagnetic radiation, as to induce the production of microbubbles; and
(c) exposing the microbubbles of step (b) to ultrasound radiation;
wherein said microbubbles emit heat upon exposure to the ultrasound radiation.

According to certain embodiments, the nanoparticles are designed to form clusters characterized by enhanced photothermal interaction cross section with an electromagnetic radiation.

According to other embodiments, the particles can be made of metal or of non-metallic material like carbon. The particles can be coated with materials which enhance their tendency to form clusters. The dimension of the particles is typically on a scale of a few tens to about one thousand nanometer, and they can have any desired external shape including spherical, cubic, oval and rod shapes. The structure of the nanoparticles can be solid, core/shell, hollow, tubular or star-like. According to certain embodiments, the nanoparticles diameter is in the range of from about 10 nm to about 1,000 nm.

According to certain embodiments, the nanoparticles are administered to the cell or tissue in a concentration of 10⁵ to 10⁹ nanoparticles/cm³, preferably in a concentration of 3*10⁵ to 3*10⁷.

Nanoparticles administered to the cell or tissue are typically coated with materials (e.g., polyethyleneglycol) which prevent them from clustering or agglomerating. Clustering may be triggered by several mechanisms, for example by coating the nanoparticles with materials whose anti clustering action is eliminated by exposure to external stimulus, including, for example, electromagnetic radiation, ultrasound radiation and shock wave. Alternatively, nanoparticles of a second type are administered together with the administration of the radiation-absorbing nanoparticles. The second type of nanoparticles is coated with materials which upon activation by external stimulus, neutralize the anti-clustering coating of the radiation-absorbing nanoparticles and induce clustering or agglomerating.

According to further embodiments, the clustering tendency of the nanoparticles is triggered by the electromagnetic source or the ultrasound source. In a preferred embodiment the tendency for clustering is triggered by addition of complementary nanoparticles, which interact with the nanoparticles initially administered to the cell or tissue. According to yet another embodiment, the nanoparticles are designed for enhanced tendency to attach onto the targeted cells or tissue in clusters. In a currently preferred embodiment, the nanoparticles are designed for enhanced tendency to cluster in the presence of elevated metabolic activity of the targeted cells or tissue. The administered nanoparticles can be targeted to a desired location within a tissue or a body using any appropriate method as is known to a person skilled in the art. According to one embodiment, the administered nanoparticles are targeted to the desired location by the use of appropriate chemical schemes, including, for example antigen-antibody complexes and ligand-receptor complexes. In a preferred embodiment, antigen-antibody binding is used for targeting.

According to yet another embodiment, the ultrasound radiation is applied from one or more sources in a way designed to achieve appropriate focusing onto the targeted cells or tissue. Preferably, the ultrasound radiation is applied internally, using a minimally invasive applicator, for example using a suitable ultrasound source located at the distal end of a catheter. According to certain other embodiments, the electromagnetic radiation is also applied internally, using a minimally invasive dispersive light guide.

According to other embodiments, the electromagnetic and ultrasound radiation treatment is applied on cells or tissue which were previously exposed to an electric field whose parameters are optimized to sensitize the cell or tissue.

According to certain embodiments, the system and/or method are used to treat cancer. In alternative embodiments, the system and/or method are applied to treat non-malignant tumors. In either of these aspects, the method may be the sole method, or it may be used in combination with another therapy.

According to additional embodiments, the system and/or method of the present invention is utilized to dissolve blood clots, break kidney stones, or treat inflammations and undesired skin conditions.

According to yet other certain embodiments, the system and/or method are used for joining a tissue. The method of joining tissue can be used for procedures such as closure of skin wounds, vascular anastamosis, occular repair, nerve repair, cartilage repair, and liver repair. According to certain embodiments, the method is used for joining a tissue to a non-tissue material.

According to further embodiments, the system and/or method are used for cosmetic treatment of targeted skin regions. The cosmetic treatment includes, but is not limited to, treating vascular lesions, pigmented lesions, acne and unsightly skin formation; removing unwanted hair; and reducing stretch marks or wrinkles.

The present invention further provides system and methods for clear and localized imaging of cells and/or tissue using nanoparticles. The nanoparticles are administered to cells and/or tissue, and following their exposure to electromagnetic radiation generate microbubbles, which in turn enhance the ultrasound imaging contrast of the cells or tissue.

According to another aspect, the present invention provides an ultrasonic imaging system for diagnosing a cell or a tissue preloaded with nanoparticles comprising:
(a) an electromagnetic radiation source configured to irradiate the nanoparticles to induce the production of microbubbles by said nanoparticles;
(b) an imaging ultrasonic wave generating source configured to irradiate the microbubbles as to enhance the ultrasound imaging contrast of said cell or tissue administered with said nanoparticles;
(c) driving means coupled to the imaging ultrasonic wave generating source for driving said imaging ultrasonic source with a drive signal to generate imaging ultrasonic waves; and
(d) an ultrasound probe.

According to one embodiment, the preloaded nanoparticles used for diagnosis comprise metal. According to another, currently preferred embodiment, the metal nanoparticles are coated with materials which enhance their tendency to form clusters. The dimension of the particles is on a scale of tens to about 1,000 nanometers, and the electromagnetic radiation used is visible or infrared radiation.

According to certain imaging embodiment, the electromagnetic radiation is selected from the group consisting of visible radiation and infrared radiation. According to one currently preferred embodiment, the electromagnetic radiation is visible radiation. The radiation can be applied as a single or multiple light pulses, wherein the width of the pulses ranges between 0.01 and 10 microseconds.

According to certain embodiments, the imaging ultrasonic wave generating source (also defined herein as “imaging ultrasonic source”) is configured as to provide ultrasonic waves suitable for stabilizing the microbubbles and ultrasonic waves suitable for imaging of the suspected tissue. Both wave types can be produced by one source or can be provided by two separate sources.

According to one embodiment, inducing and maintaining the microbubble stability is obtained by a low intensity, continuous ultrasound radiation. According to another embodiment, the preferred emission mode for imaging is a pulse train (high repetition narrow pulses). According to one currently preferred embodiment the pulse frequency is in the range of from 1 to 3 MHz. The preferred pulse peak power is below the FDA permitted level for diagnostic imaging. According to one embodiment, the peak power is below 125 mW/cm².

According to certain embodiments, the imaging ultrasonic source is employed in the pulse sequencing (CPS) emission mode to obtain additional tissue parameters as temperature and coagulation level. According to one embodiment, the probe signal is processed in a B-mode to obtain two-dimensional image of the suspected tissue and/or the distribution of additional tissue parameters.

According to yet another aspect, the present invention provides a method for ultrasonic imaging of a cell or a tissue, comprising:
(d) administering nanoparticles to the cell or tissue;
(e) irradiating the nanoparticles administered to said cell or tissue by electromagnetic radiation, as to induce the production of microbubbles; and
(f) exposing the microbubbles of step (b) to ultrasound radiation;
wherein said microbubbles enhance the ultrasound imaging contrast of said cell or tissue administered with said nanoparticles.

According to one embodiment, the ultrasonic imaging method of the present invention is utilized for diagnosing a diseased cell or tissue surrounded by healthy cells or tissue.

According to another embodiment, the ultrasonic imaging method of the present invention is utilized for imaging during a therapeutic treatment.

Other objects, features and advantages of the present invention will become clear from the following description and drawings.

### BRIEF DESCRIPTION OF THE FIGURES

FIG. 1 illustrates the operation range of the present invention on the ultrasound power density—microbubble diameter plane.

FIG. 2 shows a preferred embodiment of a combined electromagnetic radiation—ultrasound radiation therapeutic apparatus for treating a target tissue located near a subject surface.

FIG. 3 depicts the stages of the combined treatment leading to the localized release of heat within the targeted tissue volume.

FIG. 4 shows the stages of microbubble inception and stabilization by the combined action of electromagnetic and ultrasound radiation on nanoparticle clusters attached to a target cell.

FIG. 5 shows a preferred embodiment of a combined electromagnetic radiation—ultrasound radiation therapeutic apparatus for treating a target tissue located deep within a subject body.

FIG. 6 illustrates a preferred embodiment of a combined electromagnetic radiation—ultrasound radiation diagnostic imaging apparatus for imaging a target tissue located near a subject surface.

FIG. 7 shows a preferred embodiment of a combined electromagnetic radiation—ultrasound radiation diagnostic imaging apparatus for imaging a target tissue located deep within a subject body.

FIG. 8 depicts a preferred embodiment for the combined electromagnetic radiation—ultrasound radiation therapeutic apparatus operating in the ultrasound guided treatment mode.

### DETAILED DESCRIPTION OF THE INVENTION

### Definitions

As used herein, “energy source” encompasses any and all forms of excitation, including radiation from any or all regions of the electromagnetic spectrum, ultrasound, magnetic fields, electric fields, microwave radiation, laser radiation, etc.

As used herein, “light” means electromagnetic radiation.

As used herein, “electromagnetic radiation” is defined as radiation having an electric field and a magnetic field propagating at right angles to one another and is further limited to only the following: microwaves, infrared, visible, ultraviolet, x-rays, gamma rays, and cosmic rays. As used herein, “electromagnetic radiation” does not include radio-frequency radiation.

As used herein, “nanoparticle” is defined as a particle having a diameter of from 1 to 1000 nanometers, having any size, shape, structure or morphology exhibiting enhanced absorption of electromagnetic radiation in a relatively narrow spectral band, between 300 nm and 2000 nm, as a single particle or as a cluster or an agglomerate of nanoparticles.

As used herein “delivering” nanoparticles to a location is defined as affecting the placement of the nanoparticles attached to, next to, or sufficiently close to the location such that any heat generated by the microbubbles generated from the nanoparticles is transferred to the location and any imaging of the local environment by the includes imaging of the desired location.

The term “targeted” as used herein encompasses the use of antigen-antibody binding, ligand-receptor binding, and other chemical binding interactions, as well as non-chemical means such as direct injection.

As used herein, “cluster” is defined as a plurality of nanoparticles spread on a surface of a tissue. The term “agglomerate” is defined as a plurality of nanoparticles agglomerated in a 3-dimensional structure.

The term “tumor” as used herein includes any swelling or tumefaction. As used herein, tumor also refers to a neoplasm.

The term “benign tumor” as used herein is defined as a tumor that does not form metastases and does not invade or destroy adjacent tissue. The term “malignant tumor” as used herein is defined as a tumor that invades surrounding tissues, is usually capable of producing metastases and may recur after attempted removal. The term “cancer” as used herein is defined as a general variety of malignant neoplasms.

The term “antibody” as used herein, refers to an immunoglobulin molecule, which is able to specifically bind to a specific epitope on an antigen. As used herein, an antibody is intended to refer broadly to any immunologic binding agent such as IgG, IgM, IgA, IgD and IgE. Antibodies can be intact immunoglobulins derived from natural sources or from recombinant sources and can be immunoactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies.

The term “autoimmune disease” as used herein is defined as a disorder that results from autoimmune responses. Autoimmunity is an inappropriate and excessive response to self-antigens. Examples include but are not limited to, Addision's disease, Graves' disease, multiple sclerosis, myxedema, pernicious anemia, rheumatic fever, rheumatoid arthritis, systemic lupus erythematosus, and ulcerative colitis.

The term “inflammation” as used herein, is a general term for the local accumulation of fluid, plasma proteins, and white blood cells that is initiated by physical injury, infection or a local immune response. This is also known as an inflammatory response. The cells that invade tissue undergoing inflammatory responses are often called inflammatory cells or an inflammatory infiltrate.

As used herein, “localized” means substantially limited to a desired area with only minimal, if any, dissemination outside of such area.

### PREFERRED MODE OF THE INVENTION

The system and methods of the present invention are suitable for highly localized, targeted, and minimally invasive treatment strategies based on microbubble generation due to photothermal interactions of electromagnetic radiation with nanoparticles, and then exposing the microbubbles to ultrasound radiation. Any standard method can be applied for administering the nanoparticles to a desired cell or tissue of an animal. Animals that may be treated using the method of the invention include, but are not limited to humans, cows, horses, pigs, dogs, cats, sheep goats, rabbits, rats, mice, birds or chickens.

The system and methods of the present invention are not restricted to a specific type of nanoparticles, providing the nanoparticles can be administered to a cell or a tissue and have significant electromagnetic radiation absorption cross section. For example, the nanoparticles may comprise dielectic core and metal shell, they could have a cage structure or have a rod or tubular shape. The nanoparticles size ranges from a few nm to one micron.

Electromagnetic Radiation Absorbing Nanoparticles

Electromagnetic radiation absorbing nanoparticles typically include a metal component that could be gold, silver, copper, platinum, palladium, lead, and iron but also could be made from non-metallic materials, for example carbon. Gold is typically most preferred. Gold nanoparticles are commonly used in biological and biomedical applications because of their inertness under physiological conditions. They are also well known for their intense absorption and scattering properties. Gold nanoparticles functionalized with biomolecular ligands have been employed as carriers and labels in biological tissue staining, drug and gene delivery, and biosensing applications. In this regard, any metallic particle may be coated with gold. The optical responses of colloidal gold particles are enhanced by collective electronic excitations known as surface plasmons, which are responsible for absorption cross section which is similar or higher compared to the physical cross section of the nanoparticle [Yguerabide J, and Yguerabide E. E. I. Theory. Anal Biochem. 262:137-56, (1998)]. Gold nanoparticles can have anisotropic absorption properties, which vary with respect to their orientation relative to the incident optical radiation.

Since nanoparticle resonance decays nonradiatively (with typical quantum efficiencies of a few percent), most of the energy due to optical absorption is converted into heat. Thus resonant illumination of highly absorptive nanoparticles can provide significant local heating to the microscopic environment of the nanoparticles. Furthermore, their optical emissions do not bleach over time and have no saturation limits.

Gold nanoshells are a type of metal nanoparticle composed of a dielectric (for instance, silica) core coated with one or more gold shell layers. Gold nanoshells possess physical properties similar to gold colloid, in particular, a strong optical absorption due to the collective electronic response of the gold to light. The spectral location of the maximum plasmon resonance peak depends upon the ratio of the core radius to shell thickness, as well as upon the dielectric functions of the core and shell. The presence of a dielectric core shifts the plasmon resonance to longer wavelengths relative to a solid nanoparticle made exclusively of the gold shell material.

Recent activities in nanoscale material science have further expanded the knowledge base regarding the optical physics of metallic nanoparticles, and it is now evident that physical structure has a dramatic influence on plasmon-enhanced response. In particular, the optical resonance of anisotropic metallic nanoparticles such as rods, ellipsoids, and triangles have been found to be more intense and frequency-specific than their spherical counterparts, and can be tuned as a function of their size, shape, and interparticle coupling [Jensen T. et al. J. Cluster Sci. 10:295-317, (1999); El-Sayed, M. A. Acc. Chem. Res. 34:257-64, (2001)].

An important feature of plasmon resonance is its high sensitivity to shape anisotropy: isolated symmetrical nanoparticles typically support a single resonance frequency, whereas anisotropic particles (rods, triangles, ellipsoids, etc.) will exhibit at least one additional plasmon mode. In the case of cylindrical nanorods, the frequency of this second (longitudinal) plasmon mode is determined primarily by the particle's aspect ratio, and is red-shifted well into the NIR. It has been shown, both theoretically and experimentally, that gold nanorods with aspect ratios of 4:1 exhibit longitudinal plasmon resonance centered at 800 nm, whereas nanorods with aspect ratios of 9:1 exhibit resonance centered at 1.3μ [Jensen T. et al supra; El-Sayed, M., supra; Yu Y. Y., et al., J. Phys. Chem. B 101:6661-6664, (1997)]. One type of high aspect ratio nanoparticles is nanotubes, which are typically made of carbon. Similarly to nanorods, they also absorb light in two resonant wavelengths, where the longitudinal one is shifted towards the IR. Carbon nanotubes are capable of carrying therapeutic substances in their hollow volume. This payload may be released by heating the nanotube.

The extremely agile “tunability” of the optical resonance is a property completely unique to nanoparticles. This spectral tenability region includes the 800-1300 nm and 1600-1850 nm “water windows” of the near infrared, a region of high physiological transmittance which has been demonstrated as the spectral region best suited for optical bio-imaging and bio-sensing applications. The spectral full width at half maximum (FWHM) can also be varied as a function of the nanoparticles size. Generally, higher peak absorption cross section would result in reducing the spectral FWHM.

Preparation of Electromagnetic-Radiation Absorbing Nanoparticles

Nanoparticles of the nanoshell type may be prepared as described in U.S. Pat. No. 6,530,944 to West et al. Briefly, nanometer sized gold particles are added to a dispersion of silica spheres (core) to form a seed on the core surface with the presence of organosilane linker. Additional gold is deposited to the seeded core surfaces using chemical reduction reaction (e.g., from HAuCl₄). Finally, the nanoparticles dispersion is rinsed from the chemicals, leaving clean gold nanoshell dispersion.

Nanoparticles of the nanocage type may be prepared as described in Chen J. et al., [Chen J. et al. Nanoletters, 5 (3) p. 473-477 (2005)]. The process involves mixing of poly(vinyl pyrridone) with a dispersion of pre-prepared silver nanocubes. A solution of HAuCl₄ is added slowly until the color becomes stable, stirred vigorously and cooled to room temperature. The dispersion is then rinsed with saturated NaCl solution to dissolve the AgCl precipitate and rinsed again leaving clean gold nanocage dispersion.

Loading Nanoparticles into a Cell or a Tissue

The nanoparticles of the present invention may be administered to the cell or tissue using targeting schemes involving specific chemical interactions (e.g., antigen-antibody binding, etc.) or may consist of the simple delivery of the nanoparticles to the desired area, preferably by the delivery of a pharmaceutical composition comprising the nanoparticles according to the present invention. The direction or targeting of the therapy may be to the surface of the subject cells and/or tissue, or it may be to other, interior sites.

Various types of pharmaceutical compositions can be used according to the teaching of the present invention, depending on the desired form of administration.

Aqueous compositions comprise an effective amount of nanoparticles dissolved and/or dispersed in a pharmaceutically acceptable carrier and/or aqueous medium. As used herein, the terms “pharmaceutically and/or pharmacologically acceptable” refer to molecular entities and/or compositions that do not produce an adverse, allergic and/or other deleterious effects when administered to an animal, as appropriate. As used herein, “pharmaceutically acceptable carrier” includes, but is not limited to solvents, dispersion media, coatings, antibacterial agents, antifungal agents, isotonic and/or absorption delaying agents and the like. The use of pharmaceutically acceptable carrier is well known in the art. The pharmaceutical composition can further comprise supplementary active ingredients.

According to certain embodiments, the pharmaceutical composition is formulated for parenteral administration, e.g., formulated for injection via the intravenous, intramuscular, sub-cutaneous, intralesional, and intraperitoneal routes. Typically, such compositions are prepared either as liquid solutions or suspensions; solid forms suitable for using to prepare solutions and/or suspensions upon the addition of a liquid prior to injection can also be prepared; and the preparations can also be emulsified.

The nanoparticle compositions of the present invention can be formulated into a composition in a neutral and/or salt form. Any pharmaceutically acceptable salt known to a person skilled in the art can be used, providing it would not interfere with the function of the nanoparticles.

Sterile injectable solutions are prepared by incorporating the active compounds, specifically the nanoparticles in the required amount in the appropriate solvent with other ingredients as detailed above, as required, followed by filtered sterilization. Generally, dispersions are prepared by incorporating the various sterilized active ingredients into a sterile vehicle which contains the basic dispersion medium and/or the required other ingredients as described herein above. In the case of sterile powders for the preparation of sterile injectable solutions, the preferred methods of preparation are vacuum-drying and/or freeze-drying techniques which yield a powder of the active ingredient plus any additional desired ingredient from a previously sterile-filtered solution thereof. The preparation of more, and/or highly, concentrated solutions for direct injection is also contemplated, where the use of DMSO as solvent is envisioned to result in extremely rapid penetration, delivering high concentrations of the active agents to a small target area.

Upon formulation, solutions are administered in a manner compatible with the dosage formulation and/or in such amount as is therapeutically effective. The formulations are easily administered in a variety of dosage forms, such as the type of injectable solutions described above, but drug release capsules and/or the like can also be employed.

Other pharmaceutically acceptable forms of nanoparticles composition include, for example, tablets and/or other solids for oral administration; liposomal formulations; time release capsules; and/or any other form currently in use, including creams. One may also use nasal solutions and/or sprays, aerosols and/or inhalants compositions of nanoparticles of the present invention. Nasal solutions are usually aqueous solutions designed to be administered to the nasal passages in drops and/or sprays.

Additional formulations which are suitable for other modes of administration include vaginal suppositories and/or pessaries. A rectal pessary and/or suppository may also be used. Suppositories are solid dosage forms of various weights and/or shapes, usually medicated, for insertion into the rectum, vagina and/or the urethra. After insertion, suppositories soften, melt and/or dissolve in the cavity fluids. In general, for suppositories, traditional binders and/or carriers may include, for example, polyalkylene glycols and/or triglycerides.

Other delivery methods of the present invention comprise compositions comprising one or more lipids associated with at least one nanoparticle. A lipid is a substance that is characteristically insoluble in water and extractable with an organic solvent. Lipids include, for example, the substances comprising the fatty droplets that naturally occur in the cytoplasm as well as the class of compounds which are well known to those of skill in the art which contain long-chain aliphatic hydrocarbons and their derivatives, such as fatty acids, alcohols, amines, amino alcohols, and aldehydes. The above-described examples are not meant to be limiting, and compounds other than those specifically described herein that are understood by one of skill in the art as lipids are also encompassed by the compositions and methods of the present invention.

A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof.

In particular embodiments, a lipid comprises a liposome. A liposome is a generic term encompassing a variety of single and multilamellar lipid vehicles formed by the generation of enclosed lipid bilayers or aggregates. Liposomes may be characterized as having vesicular structures with a bilayer membrane, generally comprising a phospholipid, and an inner medium that generally comprises an aqueous composition.

A multilamellar liposome has multiple lipid layers separated by aqueous medium. They form spontaneously when lipids comprising phospholipids are suspended in an excess of aqueous solution. The lipid components undergo self-rearrangement before the formation of closed structures, entrapping water and dissolved solutes between the lipid bilayers. Lipophilic molecules or molecules with lipophilic regions may also dissolve in or associate with the lipid bilayer.

In particular embodiments, a nanoparticle may be, for example, encapsulated in the aqueous interior of a liposome, interspersed within the lipid bilayer of a liposome, attached to a liposome via a linking molecule that is associated with both the liposome and the nanoparticle, entrapped in a liposome, complexed with a liposome, etc.

A liposome used according to the present invention can be made by different methods, as would be known to one of ordinary skill in the art. Phospholipids can form a variety of structures other than liposomes when dispersed in water, depending on the molar ratio of lipid to water. At low ratios the liposome is the preferred structure.

The size of a liposome varies depending on the method of synthesis. Liposomes in the present invention can have a variety of sizes. In certain embodiments, the liposomes are small, e.g., less than about 100 μm, about 90 μm, about 80 nm, about 70 μm, about 60 nm, or less than about 50 nm in external diameter. In preparing such liposomes, any protocol described herein, or as would be known to one of ordinary skill in the art may be used. Additional non-limiting examples of preparing liposomes are described in U.S. Pat. Nos. 4,728,575, 4,737,323, 4,533,254, 4,162,282, 4,310,505, and 4,921,706; A comprehensive review of lipid vesicles and methods for their preparation are described in “Liposome Technology” (1984. Gregoriadis G. ed. CRC Press Inc Boca Raton Fla. Vol I II & III).

Liposomes interact with cells to deliver agents via four different mechanisms: Endocytosis by phagocytic cells of the reticuloendothelial system such as macrophages and/or neutrophils; adsorption to the cell surface, either by nonspecific weak hydrophobic and/or electrostatic forces, and/or by specific interactions with cell-surface components; fusion with the plasma cell membrane by insertion of the lipid bilayer of the liposome into the plasma membrane, with simultaneous release of liposomal contents into the cytoplasm; and/or by transfer of liposomal lipids to cellular and/or subcellular membranes, and/or vice versa, without any association of the liposome contents. Varying the liposome formulation can alter which mechanism is operative, although more than one may operate at the same time.

According to certain embodiments, ligands are added to the liposomes to facilitate the delivery of the nanoparticle-containing liposomes to the desired cell or tissue. Targeted delivery is achieved by the addition of ligands without compromising the ability of these liposomes to deliver large amounts of nanoparticles. It is contemplated that this will enable delivery to specific cells, tissues and organs. The targeting specificity of the ligand-based delivery systems is based on the distribution of the ligand receptors on different cell types. The targeting ligand may either be non-covalently or covalently associated with the lipid complex, and can be conjugated to the liposomes by a variety of methods.

The targeting ligand can be either anchored in the hydrophobic portion of the complex or attached to reactive terminal groups of the hydrophilic portion of the complex. The targeting ligand can be attached to the liposome via a linkage to a reactive group, e.g., on the distal end of the hydrophilic polymer. Preferred reactive groups include amino groups, carboxylic groups, hydrazide groups, and thiol groups. The coupling of the targeting ligand to the hydrophilic polymer can be performed by standard methods of organic chemistry that are known to those skilled in the art. In certain embodiments, the total concentration of the targeting ligand can be from about 0.01 to about 10% mol.

Targeting ligands are any ligand specific for a characteristic component of the targeted region. Preferred targeting ligands include proteins such as polyclonal or monoclonal antibodies, antibody fragments, or chimeric antibodies, enzymes, or hormones, or sugars such as mono-, oligo- and poly-saccharides. For example, disialoganglioside GD2 is a tumor antigen that has been identified in neuroectodermal origin tumors, such as neuroblastoma, melanoma, small-cell lung carcinoma, glioma and certain sarcomas. Liposomes containing anti-disialoganglioside GD2 monoclonal antibodies have been used to aid targeting of the liposomes to cells expressing the tumor antigen. In another non-limiting example, breast and gynecological cancer antigen specific antibodies are described in U.S. Pat. No. 5,939,277. In a further non-limiting example, prostate cancer specific antibodies are disclosed in U.S. Pat. No. 6,107,090. Thus, it is contemplated that the antibodies as would be known to one of ordinary skill in the art may be used to target the nanoparticles of the present invention to specific tissues and cell types. In certain embodiments of the invention, contemplated targeting ligands interact with integrins, proteoglycans, glycoproteins, receptors or transporters. Suitable ligands include any that are specific for cells of the target organ, or for structures of the target organ exposed to the circulation as a result of local pathology, such as tumors.

In certain embodiments of the present invention, in order to enhance the transduction of cells, to increase transduction of target cells, or to limit transduction of undesired cells, antibody or cyclic peptide targeting moieties (ligands) are associated with the lipid complex. Such methods are known in the art. For example, liposomes that specifically target cells of the mammalian central nervous system have been described in U.S. Pat. No. 5,786,214. The liposomes are composed essentially of N-glutarylphosphatidylethanolamine, cholesterol and oleic acid, wherein a monoclonal antibody specific for neuroglia is conjugated to the liposomes. It is contemplated that a monoclonal antibody or antibody fragment may be used to target delivery to specific cells, tissues, or organs in the animal, such as for example, brain, heart, lung, liver, etc.

Still further, a nanoparticle may be delivered to a target cell via receptor-mediated delivery and/or targeting vehicles comprising a lipid or liposome. These take advantage of the selective uptake of macromolecules by receptor-mediated endocytosis that will be occurring in a target cell. In view of the cell type-specific distribution of various receptors, this delivery method adds another degree of specificity to the present invention.

Thus, in certain aspects of the present invention, a ligand will be chosen to correspond to a receptor specifically expressed on the target cell population. A cell-specific nanoparticle delivery and/or targeting vehicle may comprise a specific binding ligand in combination with a liposome. The nanoparticle to be delivered are housed within a liposome and the specific binding ligand is functionally incorporated into a liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the specific binding ligand may comprise one or more lipids or glycoproteins that direct cell-specific binding. For example, U.S. Pat. No. 5,432,260 discloses that the sugars mannosyl, fucosyl or N-acetyl glucosamine, when coupled to the backbone of a polypeptide, bind the high affinity manose receptor. It is contemplated that the nanoparticles of the present invention can be specifically delivered into a target cell or tissue in a similar manner.

Folate and the folate receptor have also been described as useful for cellular targeting (U.S. Pat. No. 5,871,727). In this example, the vitamin folate is coupled to the complex. The folate receptor has high affinity for its ligand and is overexpressed on the surface of several malignant cell lines, including lung, breast and brain tumors. Anti-folate such as methotrexate may also be used as targeting ligands. Transferrin mediated delivery systems target a wide range of replicating cells that express the transferrin receptor.

A skilled artisan realizes that the systems and methods of the present invention can be employed in a variety of types of experimental, therapeutic and diagnostic procedures, including in vitro or in vivo experimental procedures.

The systems and methods of the present invention can be applied to a cell or a tissue, wherein the cell can be part of a tissue, such as a tumor tissue.

In certain embodiments, a cell may comprise, but is not limited to, at least one skin, bone, neuron, axon, cartilage, blood vessel, cornea, muscle, facia, brain, prostate, breast, endometrium, lung, pancreas, small intestine, blood, liver, testes, ovaries, cervix, colon, skin, stomach, esophagus, spleen, lymph node, bone marrow, kidney, peripheral blood, embryonic or ascite cell, and all cancers thereof.

In further embodiments, a tissue may comprise a cell or cells to be transformed with a nanoparticle of the present invention. The tissue may be part or separated from an organism. In certain embodiments, a tissue may comprise, but is not limited to, adipocytes, alveolar, ameloblasts, axon, basal cells, blood (e.g., lymphocytes), blood vessel, bone, bone marrow, brain, breast, cartilage, cervix, colon, cornea, embryonic, endometrium, endothelial, epithelial, esophagus, facia, fibroblast, follicular, ganglion cells, glial cells, goblet cells, kidney, liver, lung, lymph node, muscle, neuron, ovaries, pancreas, peripheral blood, prostate, skin, small intestine, spleen, stem cells, stomach, testes or ascite tissue, and all cancers thereof.

Additional in vivo assays involve the use of various animal models, including transgenic animals that have been engineered to have specific defects, or carry markers that can be used to measure the ability of the systems and methods of the present invention to effect different cells or tissues within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons).

Microbubbles Production by Nanoparticles

According to the teaching of the present invention, the loaded nanoparticles are first irradiated by electromagnetic radiation to produce microbubbles.

When an absorbing particle (nanoparticle, a cluster or an agglomerate of absorbing nanoparticles) is exposed to a continuous electromagnetic radiation, the absorbed power is transferred to the surrounding tissue thereby increasing the tissue temperature. However, when the same absorbing particle is exposed to pulse electromagnetic radiation, its temperature increases momentarily and than decays as the heat diffuses to a small region around it. Above certain electromagnetic power flux, the particle temperature exceeds sufficient threshold, and in turn evaporates a small liquid region around it in the form of a cavitation microbubble.

For sufficiently short pulse heating, the peak particle temperature increase, ΔT, is expressed as

$$Δ T = \frac{3 P σ {t}_{p}}{4 πρ {C}_{p} {r}_{m}^{3}} \qquad ( 2 )$$

Where P and tₚ are the radiation power and pulse width and a is the particle absorption cross section for the radiation wavelength, and ρ, Cp and rₘ are the particle density, heat capacity and radius (or half thickness) respectively. Following the pulse, the particle temperature decays exponentially with a constant that is highly dependent upon the particle size and also on the particle-liquid film heat transfer coefficient.

However, the actual particle temperature rise is much lower, due to thermal conduction to the surrounding liquid. The actual temperature rise depends upon the electromagnetic radiation pulse width and the particle radius. The time dependent particle relative temperature can be calculated from the following ordinary differential equation:

$$\frac{ⅆ T}{ⅆ t} = \frac{Δ T}{{τ}_{p}} - \frac{T}{{t}_{s}} where \\ {t}_{s} = \frac{4 {r}_{m}^{2}}{27 α} α = \frac{k}{ρ {C}_{p}} \qquad ( 3 )$$

and where k and α are the water conductivity, and thermal diffusivity, respectively, tₛ is the relative temperature decay time to 1/e of its original level, and T is the particle relative temperature above its surrounding. For 100 nm gold nanoparticles, the relaxation time tₛ is about 10 nsec. It is easy to show that the electromagnetic pulse heating is effective when tₚ<tₛ.

Due to the liquid pressure and surface tension, the minimum particle temperature required for microbubble nucleation is well above 100° C. It has been shown, for example, that the required peak melanosome particle temperature at that threshold for microbubble formation is about 200° C. For nanoparticles, the threshold nucleation temperature has been found to be 150° C.

Solving equation (3) for the threshold temperature can predict the minimal electromagnetic pulse energy density required for microbubble nucleation. The pulse energy density decreases with decreasing tₚ. If the components of equation (3) are examined, it appears that the first term increases as 1/rₘ but the second term increases as 1/rₘ². In other words, the peak of a laser power required for nucleation may increase even as 1/rₘ². Indeed, Zharov et al., [Zharov V. P., et al., J. Phys. D: Appl. Phys. 38:2571-2581, (2005)] found experimentally that microbubble generation from isolated nanoparticles whose size is relevant to the present invention, requires laser pulses whose energy density ranges between 3-50 J/cm² and thus is impractical for treatment applications.

Loo et al. [supra] have found that nanoparticles tend to form clusters on tumor tissues. The typical nanoparticle content of a cluster ranges between 5-50 adjacent particles. The typical distance between clusters is measured as a few microns.

Nanoparticles clusters have a very high ratio of ΔT/tₛ compared to spherical solid nanoparticle with similar mass. For example, the ΔT/tₛ for a 27 100-nm nanoparticles cluster is three times the respective ratio for a single 300 nm nanoparticle. This means that microbubbles can be generated from small nanoparticles arranged in clusters. The present invention is based in part of this property of nanoparticle clusters, since only nanoparticles whose size is below 100 nm can easily diffuse through the malignant blood vessels and certain 40 nm nanoparticles can even diffuse into cells.

Zharov et al [supra] have found that repeatable nucleation threshold is about 500 mJ/cm² for 8 nsec 633 nm laser pulse. This threshold decreases with decreasing inter-particle distance within the cluster below the 1-2 micron level. They also found that the threshold decreases with increasing tissue temperature.

The ability of low power ultrasound radiation to stabilize momentary generated microbubbles depends upon the microbubble diameter and lifetime. The generated microbubble diameter (assuming no heat losses and 100% nanoparticles heat to vapor conversion) can be calculated from the following equation:

$${D}_{v} = {[ \frac{{n}_{p} δ T ρ {C}_{p} {r}_{m}^{3}}{8 {ρ}_{v} {λ}_{v}} ]}^{1 / 3} \qquad ( 4 )$$

Where nₚ, εT are the number of nanoparticles in the cluster and their relative temperature above about 100° C., ρᵥ, λᵥ are the vapor density and latent heat, respectively. The actual peak microbubble size is significantly lower due to its quick vapor condensation. The microbubble lifetime depends on its volume to surface area ratio (i.e. rₘ) and also on the content of non-condensable gasses, which strongly affect the vapor condensation rate on the microbubble shell.

Brennen [Brennen C. E., Cavitation and Bubble Dynamics, Chapter 4: Dynamics of oscillating bubbles, Oxford University Press, (1995)] reviewed large number of publications in the field of microbubble growth and derived an expression for the threshold ultrasound pressure required to stabilize bubbles. His expressions fit experimental data for a wide range of bubbles diameters. For example, a 5-micron diameter air bubble in water, can be stabilized using ultrasound peak pressure of about 0.7 Bar or 100 mW/cm² at 2 MHz. The growth of resonance frequency microbubbles at low power ultrasound radiation is very slow and may take tens of seconds. The absorption cross-section of smaller microbubbles falls sharply. For example, at 5 MHz the resonant microbubble size is 4 micron while the cross section falls by 50% from 4πr_{b}² for 3-micron microbubble.

Thus, 3 MHz low power ultrasound radiation will be sufficient to stabilize short lifetime microbubbles whose size range from 4 to 7 microns. Exposing nanoparticles cluster to electromagnetic pulse generates 1-2 microns transient microbubbles which still have significant interaction cross section for MHz ultrasound radiation. Thus, the required ultrasound peak pressure for stabilization would be about 0.1 MPa, i.e., low power ultrasound radiation.

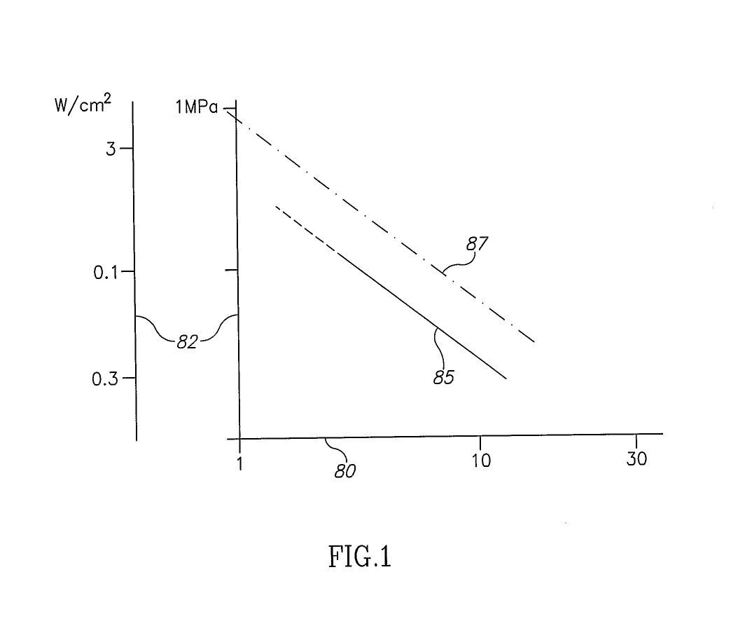

FIG. 1 depicts a plot of the threshold ultrasound peak pressure required for microbubble stabilization as a function of the microbubble radius. This figure, consists of two curves versus the microbubble diameter coordinate 80: The threshold for slowly produced air microbubble stabilization 85 is denoted in solid line and the threshold for transient mostly vapor microbubbles 87 is denoted in dash-dot line. The ultrasound power density at 2 MHz coordinate 84 is depicted in parallel to the corresponding peak pressure coordinate 82.

Thus, the present invention now discloses systems and methods for localized delivery of heat to a target cell or tissue using low power ultrasound radiation. Exposing a targeted tissue after it has been administered with nanoparticles to short electromagnetic pulses generates transient microbubbles cloud. Exposing the microbubble cloud to low power ultrasound radiation stabilizes it and at the same time couples the ultrasound power to the host targeted cell or tissue. According to one embodiment the nanoparticles are designed to release carried non-condensable gases or to generate non-condensable gases from the adjacent tissue. Thus, exposing the nanoparticles to electromagnetic pulse generates microbubble with certain non-condensable gas content. The vapor condensation rate on such microbubble wall is significantly lower compared to vapor only microbubbles. In turn, much lower ultrasound power densities will achieve the stabilization of such microbubbles.

Ultrasound Radiation

As used herein, the term “ultrasound” refers to a form of energy which consists of mechanical vibrations, the frequencies of which are so high they are above the range of human hearing. Lower frequency limit of the ultrasonic spectrum may generally be taken as about 20 kHz. Most diagnostic applications of ultrasound employ frequencies in the range 1 to 15 MHz' [Wells P. N. T. ed., Ultrasonics in Clinical Diagnosis, 2nd. Edition, Publ. Churchill Livingstone, Edinburgh, London & NY, (1977)]. The term “ultrasound” as used in this specification is intended to encompass diagnostic, therapeutic and focused ultrasound.

Ultrasound radiation, like light, can be focused very accurately on a target. Ultrasound radiation can be focused more deeply into tissues than light, and is therefore better suited for applications that require penetration into a whole tissue or a whole organ. Another important advantage of ultrasound is its non-invasive stimulus, which is used in a wide variety of diagnostic and therapeutic applications. By way of example, ultrasound is well known in medical imaging techniques and, additionally, in orthopedic therapy. Instruments suitable for the application of ultrasound to a subject vertebrate are widely available and their use is well known in the art.

Ultrasound has been used in both diagnostic and therapeutic applications. When used for imaging as a diagnostic tool, ultrasound is typically applied in an energy density of up to about 100 mW/cm² (FDA recommendation), although energy densities of up to 750 mW/cm² have also been used. In physiotherapy, ultrasound is typically used as an energy source in a density of up to about 3 to 4 W/cm² (WHO recommendation). In other therapeutic applications, higher intensities of ultrasound may be employed. Such intensities are attained by focusing the ultrasound radiation.

Focused ultrasound allows thermal energy to be delivered without an invasive probe. Another form of focused ultrasound is high intensity focused ultrasound (HIFU) (reviewed, for example, by Moussatov et al. [Moussatov et al., Ultrasonics 36(8): 893-900 (1998)].

The ultrasound radiation deposition rate increases significantly when the targeted tissue is filled with microbubbles. The scattering cross section of a single gas microbubble to ultrasound radiation is 4πR² near its resonance diameter. However, the combined attenuation and scattering effect increases the total effective attenuation. For example, Optison® contrast agent microbubble cloud (2*10⁶ bubbles/cm³, average size 2.2 micron) have a stable attenuation coefficient of 15-10 dB/cm at 3.5 MHz [Wu et al., supra]. Thus, much lower microbubble concentration of 2*10⁵ microbubbles/cm³ would be sufficient to increase the tissue absorption from 0.6 dB/cm to 2.0 dB/cm.

According to certain embodiments of the present invention, a combination of diagnostic ultrasound and a therapeutic ultrasound can be employed. This combination is not intended to be limiting, and the skilled artisan will appreciate that any variety of combinations of ultrasound may be used. Additionally, the energy density, frequency of ultrasound, and period of exposure may be varied, provided that the application of ultrasound stabilizes the microbubbles cloud and release heat.

According to additional embodiments, the ultrasound is applied to the target cell or tissue with sufficient strength to affect the cell or tissue without damaging the surrounding tissues, such that less than 10%, preferably less than 5%, more preferably less than 1% of cells within surrounding tissues are affected

According to one embodiment, the exposure to an ultrasound energy source is at a power density of from about 0.05 to about 20 W/cm², preferably from about 1 to about 15 W/cm².

According to another embodiment, the exposure to an ultrasound energy source is at a frequency of from about 0.15 to about 10.0 MHz. Preferably the exposure to an ultrasound energy source is at a frequency of from about 0.5 to about 7.5 MHz. Typically, the exposure is for periods of from about 10 milliseconds to about 60 minutes, preferably from about 1 second to about 5 minutes.

Advantageously, a target is exposed to an ultrasound energy source at an acoustic power density of from about 0.05 W/cm² to about 10 W/cm² with a frequency ranging from about 0.015 to about 10 MHz (see, for example, WO 98/52609). However, alternatives are also possible, for example, exposure to an ultrasound energy source at an acoustic power density of above 20 W/cm², but for reduced periods of time.

The ultrasound may be applied either continuously, or in the form of modulated intensity. Thus, the ultrasound may be continuous wave ultrasound or modulated pulsed wave ultrasound. According to one embodiment, the ultrasound is applied at a power density of 0.3 W/cm² to 3 W/cm² as a continuous wave. Higher power densities may be employed if pulsed wave ultrasound is used. Preferably the application of the ultrasound is in the form of multiple pulses; thus, both continuous wave and pulsed wave (pulsated delivery of ultrasound) may be employed.

According to one embodiment, the minimal required ultrasound energy density is determined by the need to stabilize the microbubbles generated by the application of electromagnetic radiation pulse on the nanoparticles that are attached to or surrounding a cell or a tissue.

According to another embodiment, the nanoparticle-loaded tissue is exposed to intermittent light pulses and continuous or modulated ultrasound radiation in order to generate and maintain dense microbubbles population within the targeted tissue. The present invention now shows that advantageously, this mode of operation enables diagnosis and therapy of a targeted tissue using light pulses and ultrasound at reduced power densities, close to the FDA regulation limits.

Nanoparticle Loading

As described herein above, various types of pharmaceutical compositions can be used for loading the nanoparticles in a target cell or tissue. The loading method is determined by the desired therapy, which may be targeted to the surface of the subject cells and/or tissue, or to other, interior sites.

According to certain embodiments, the targeted tissue is loaded with from about 10⁵ to 10⁹, preferably from about 3*10⁵ to 3*10⁷ nanoparticles/cm³. The nanoparticles are preferably attached to the tissue surfaces in clusters, typically comprising 5-50 nanoparticles per cluster. According to one embodiment, the parameters of each nanoparticle are: Diameter: 150 nm; Thermal properties: α=0.003 cm²/sec; ρ=10 gr/cm³ Cp=0.6 J/gr C; k=1.0 J/m° C.; Average cluster photothermal cross-section for NIR radiation: 5*10⁻⁹ cm².

When such typical cluster is exposed to an NIR pulse of 10 nsec, 20 mJ/cm², its temperature rises by 500° C. (assuming no conductive losses to the surrounding liquid). The cooling time constant for this cluster is estimated as 30 nsec when accounting for the film heat transfer and the cluster self-thermal shielding. However, at such energy densities, the cluster temperature exceeds 200° C., most of the deposited energy is converted to evaporation and the typical generated vapor microbubbles size reaches about 4 micron.

Clustering of the nanoparticles is desirable only after the nanoparticles reach the desired cell or tissue location; nanoparticle agglomeration or clustering in the blood stream would reduce or prevent their ability to diffuse through the blood vessels into the target cell. Any method for forming nanoparticle clusters after or during attachment to the targeted tissue can be used in the present invention. For example, clustering can be attained by releasing promoting materials with light from the nanoparticles as described, for example, in U.S. Pat. No. 6,616,946. Alternatively, one type of nanoparticles is administered to the cell or tissue and attach to the targeted tissue followed by administering a complementary type of nanoparticles coated with a substance promoting clustering to the cells or tissue to increase the nanoparticles number in each cluster.

According to certain embodiments, the nanoparticles are attached to the cell surface, either directly or via an antigen-antibody or ligand-receptor attachment mechanism. Alternative methods for retaining the nanoparticles adjacent to a targeted cell or within a targeted tissue can be also used. Radioactive dyes have been recently suggested as means for in-vivo imaging of the metabolic activity of cells or tissue, typically with Positron Emission Tomography (PET) scanning apparatus. The molecular structure of these dye materials is designed for enhanced retention time in regions of elevated metabolic activity. It is possible to use equivalent materials with high retention time and to design the nanoparticles structure, material and coating as to interact with such materials, to trigger agglomeration of the nanoparticles at regions of high concentration of these materials. This would enable nanoparticles agglomerates accumulation in regions of enhanced metabolism. According to one embodiment, the nanoparticles are designed to trigger clustering or agglomeration in the presence of chemical substances which tend to accumulate in regions of elevated metabolic activity.

Systems and Methods

Two types of systems are provided by the present invention, one type suitable for therapeutic and another type suitable for diagnostic applications. The difference between the types is the source of ultrasonic waves, wherein the therapeutic ultrasonic wave-generating source is designed to provide continuous, or pseudo continuous ultrasound radiation to be converted into heat, while for diagnostic application, an imaging ultrasonic wave-generating source provides lower intensity ultrasound radiation sufficient to maintain the microbubbles cloud.

According to one aspect, the system of the present invention comprises an electromagnetic radiation source; a therapeutic ultrasonic wave generating source and driving means coupled to the therapeutic ultrasonic wave generating source for driving said therapeutic ultrasonic source with a drive signal to generate therapeutic ultrasonic waves.

The cell or tissue to be treated by the system is first administered with nanoparticles. The electromagnetic source and the ultrasound source are operated to irradiate the cells or tissue during the treatment.

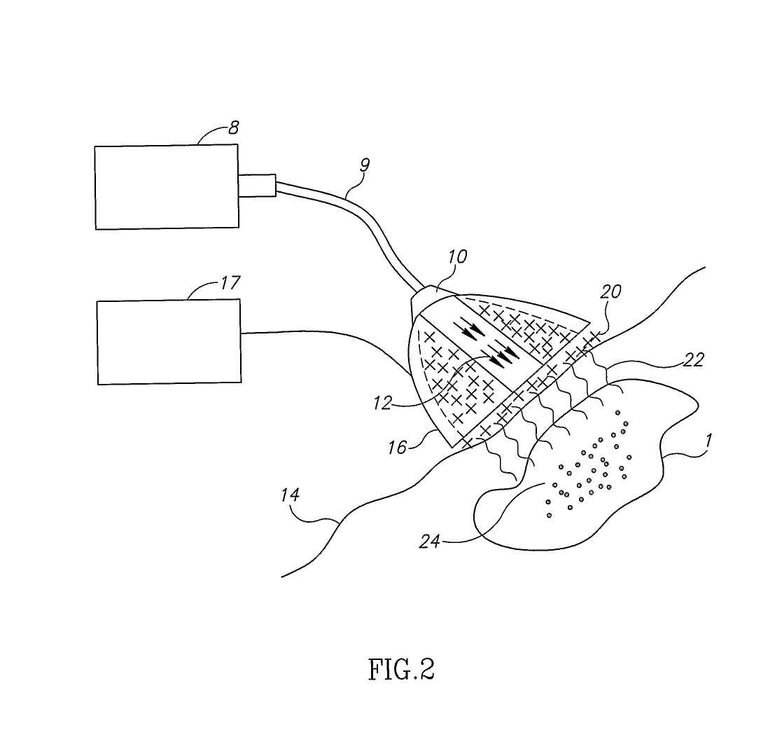

By way of illustration, a currently preferred embodiment of an apparatus suitable for treatment of tissue located within a subject body, specifically a tissue located near the outer surface of the subject body is depicted in FIG. 2. The targeted tissue 1 is administered with nanoparticles suitable for the present invention, which are designed to be attached to the tissue cells mainly as clusters or to be retained mainly in the blood vessels, as agglomerates. An electromagnetic source 8 is operable to generate electromagnetic pulse which is coupled and guided into a light guide 9 and through a coupling unit 10 and which form a sufficiently wide and uniform electromagnetic beam 12 which illuminate the targeted tissue 1 through the patient skin 14. A focusing ultrasound source 16 driven by a driving means 17, is located near the subject skin 14 and uses suitable gel 20 to couple significant portion of the ultrasound radiation 22 to the targeted tissue 1 volume illuminated by the electromagnetic source 8. Each time the nanoparticle clusters are exposed to the electromagnetic beam 12, they generate microbubble cloud 24. The microbubble cloud absorb a portion of the ultrasound radiation 22 power and convert it into heat which is emitted to the targeted tissue 1 volume. The electromagnetic source may be repetitive to conserve the desired microbubbles density within the targeted tissue 1 volume during the treatment.

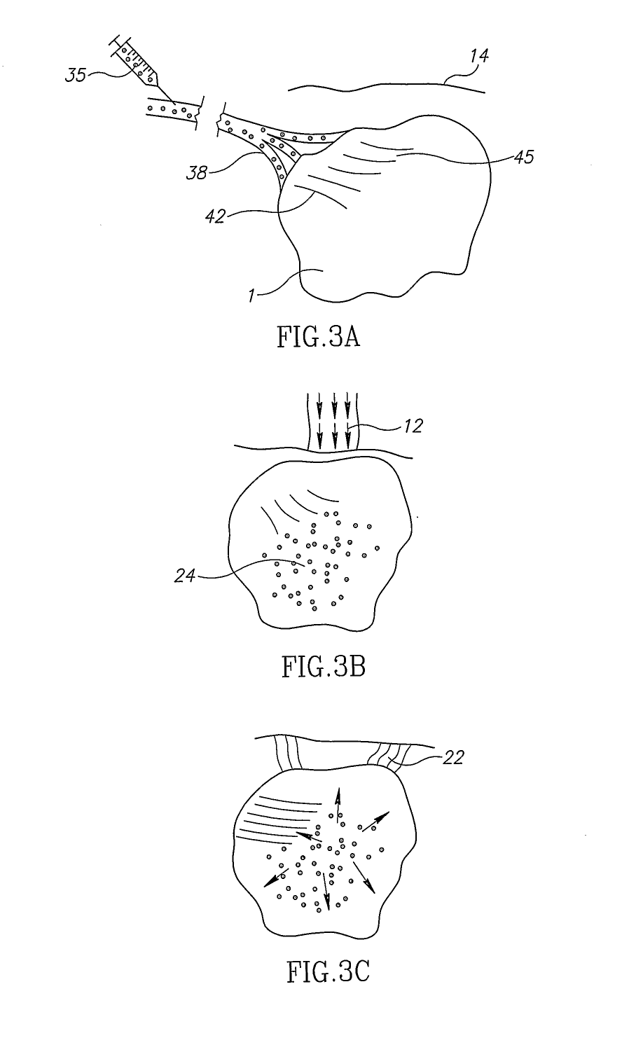

A detailed view of the targeted tissue during the treatment conducted according to one currently preferred embodiment of the present invention is illustrated in FIG. 3. In step I, the suitable nanoparticles 35 are administered to the targeted tissue 1 and accumulate within the targeted tissue blood vessels 38 and microvessels 42. Some of the nanoparticles 35 diffuse through the blood vessels 42 walls and migrate through the interstitial volume between the targeted cells 45. In turn, some of these nanoparticles stick on the targeted cells where a significant portion of them form clusters on the cell surface. Exposure of the clusters and agglomerates to pulsed electromagnetic beam 12 (step II) which penetrate the skin 14 generate microbubbles around the clusters and agglomerates which in turn form a microbubble cloud 24. The microbubble cloud 24 interact with the ultrasound radiation 22 and convert some of the ultrasound power to heat which is emitted to the surrounding targeted tissue 1 (step III). Thus, step II may be repeated in order to conserve the desired microbubble density within the targeted tissue 1 volume. In turn the electromagnetic source 8 may be operative to generate a repetitive electromagnetic pulses.

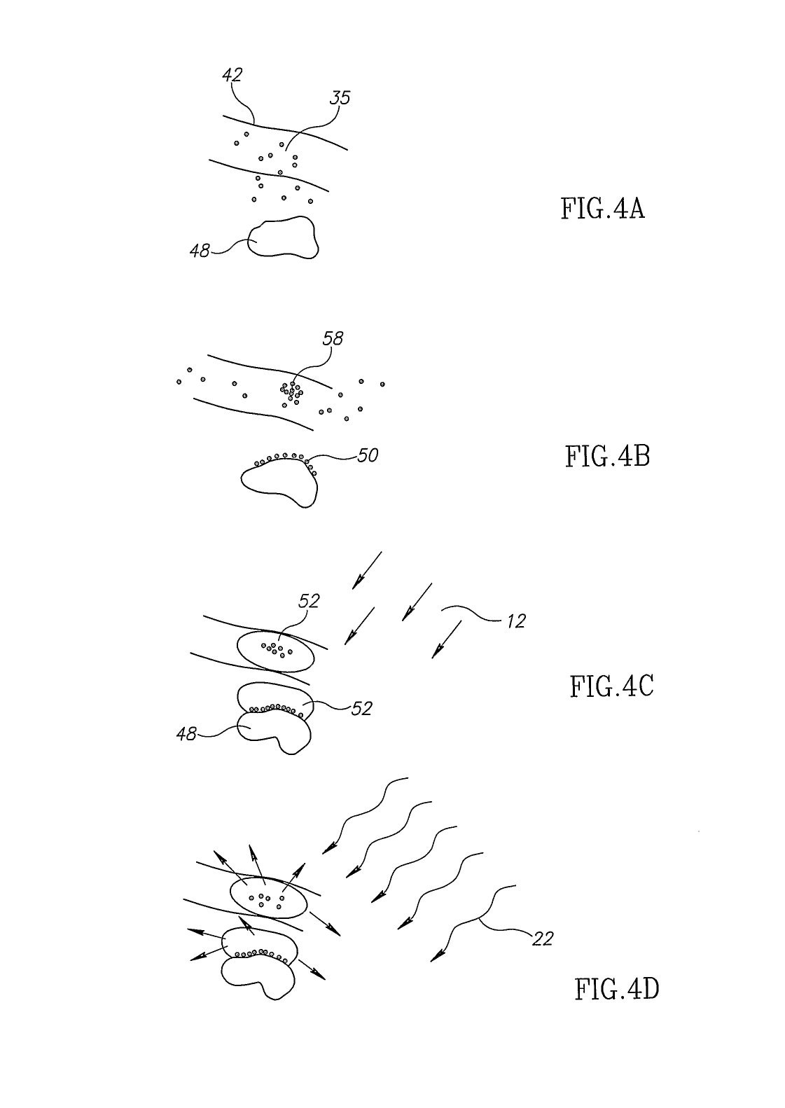

A detailed view of the targeted cells during the treatment described above is illustrated in FIG. 4. The administered suitable nanoparticles 35 penetrate through the microvessels 42 walls and accumulate on the targeted tissue cells 48 as clusters 50. When the pulsed electromagnetic beam radiation illuminates a cluster 50, the cluster absorb the radiation and converts it into heat which is released to the surrounding liquid and in turn generate boiling nucleates around each nanoparticle. The nucleates coalesce and form a transient microbubble 52 which expands rapidly to above a micron size. Each microbubble interacts with the ultrasound radiation 22 which in turn increases the microbubble 52 volume through a cyclic expansion and contraction. In turn, the microbubble 52 reaches an equilibrium average size with the ultrasound radiation while serving as an energy mediator between the ultrasound radiation 22 and the adjacent cell 48 thereby emit heat to the targeted cells. Step III may be repeated many times in order to conserve the desired microbubbles density within the targeted tissue 1 volume during the treatment.

A portion of the nanoparticles 35 administered to the targeted tissue 1 may also form agglomerates 58 within the blood microvessels 42 of the targeted tissue 1 or within the targeted tissue. Exposing each of the agglomerates 58 to pulsed electromagnetic beam 12 generates a microbubble 52 around the agglomerate 58. The microbubble interacts with the ultrasound radiation 22 very similarly to the process describes above while expanding to a certain equilibrium size. During its interaction with the ultrasound radiation 22, the microbubble 52 absorbs energy from the ultrasound and emits heat to the surrounding blood.

According to one embodiment, when the apparatus is used for therapeutic applications the light source is operated in an intermittent or pulsed mode while the therapeutic ultrasonic source can be operated continuously or at a high duty cycle. According to one currently preferred embodiment, the electromagnetic source is a pulsed infrared light source. According to another currently preferred embodiment, the ultrasound radiation frequency is between 0.5 and 7.5 MHz.

According to yet another embodiment, the ultrasound radiation source is driven by the driving means to generate an ultrasound pulse whose timing is synchronized with the electromagnetic radiation pulse so as to stabilize also the smaller microbubbles generated around the nanoparticles clusters. Next the ultrasound source is switched to continuous or modulated mode so as to induce heat emission from the microbubbles cloud.

According to another aspect, the present invention provides a method for inducing localized delivery of heat to a cell or a tissue comprising administering nanoparticles to the cell or tissue; irradiating the nanoparticles administered to said cell or tissue by electromagnetic radiation, as to induce the production of microbubbles; and exposing the formed microbubbles to ultrasound radiation; wherein said microbubbles emit heat upon exposure to the ultrasound radiation.

According to certain embodiments, system and/or method of the present invention are operated with nanoparticles suitable to generate microbubbles in an amount effective to kill or inhibit proliferation of a cancer cell. In an alternative embodiment, the method and apparatus are applied to treat non-malignant tumors. In either of these embodiments, the method may be the sole method, or it may be used in combination with another type of therapy.

According to one embodiment, the nanoparticles size may range from about ten to about 1000 nanometer and their structure is designed for enhanced cross section for the electromagnetic source. According to another embodiment, the electromagnetic radiation is infrared radiation, preferably in the range of 800 to 1300 nm. The preferred operation mode is a pulsed mode with a pulse width which ranges from 0.01 to 10 microseconds.

According to certain embodiments, the nanoparticles are metallic, their size range between 20 and 200 nm, and they are coated with a layer which after certain type of stimulation, promote clustering on the targeted cells or agglomeration with additional nanoparticles wherein the agglomerate exhibit enhanced photothermal cross section for the electromagnetic source wavelength. According to one currently preferred embodiment, the average number of nanoparticles in a cluster or agglomerate ranges between 5 and 500.

Preferably, the nanoparticles are exposed to optical pulsed radiation. Specifically, the optical radiation may be delivered to a subject through the skin surface or via a light guide inserted in a needle or endoscopes to the targeted tissue. Similarly, the ultrasound energy can be brought to the vicinity of the nanoparticles-loaded tissue with a minimally invasive catheter (see, for example, U.S. Pat. No. 5,984,882 to Rosenschein et al. which discloses a cancer therapy method based on a catheter which transfers ultrasound energy through a special metal wire). Once the nanoparticles absorb sufficient electromagnetic radiation flux, the microbubble population generated within the targeted tissue interacts with the ultrasound radiation, stabilizes and promotes effective ultrasound power deposition within the targeted tissue. The systems and methods of the present invention is advantageous over hitherto known apparatuses as the ultrasound energy would reach the targeted tissue with minimal attenuation regardless of its depth or location within the patient. The use of light/ultrasound catheters would enable hyperthermia treatments in problematic regions behind absorbing/scattering organs. These problematic regions include cancerous tissue located in the brain, prostate, lung and many other challenging locations.

Applications

Therapeutic Applications

According to the teaching of the present invention, exposing the nanoparticle clusters or agglomerates to electromagnetic radiation generates microbubbles. The interaction of ultrasound radiation with the microbubbles generates localized heat. This localized hyperthermia can be useful in a variety of clinical conditions, for example tumors (malignant or benign), inflammatory responses or autoimmune diseases. It can be used as the primary therapy, for example by killing or inhibiting the proliferation of cancer cells. Alternatively, the heat can enhance other primary therapies, for example chemotherapy or gene therapy.

To achieve cell killing or stasis, the nanoparticle, radiation and/or additional agent(s) are delivered to one or more cells generate microbubbles in an effective amount to kill the cell(s) or prevent them from dividing. Preferably, the nanoparticles are delivered to the target cell in one or more forms of pharmaceutical composition.

According to certain embodiments of the present invention, the systems and methods of the present invention are applied in combination with additional therapy. During the therapy procedure, the nanoparticles with enhanced absorption cross section are exposed to light pulses, thereby generating microbubbles. The interaction of these microbubbles with the applied ultrasound radiation releases heat to the targeted cell or tissue and also applies dynamic stress on the tissue structure. This stress enhances the penetration rate of therapeutic compositions and additional nanoparticles into the targeted tissue including to interstitial volumes. The penetration of such therapeutic compositions and nanoparticles into the targeted tissue, which were initially inaccessible to them, improves the chance for successful and complete treatment of the targeted cell or tissue.

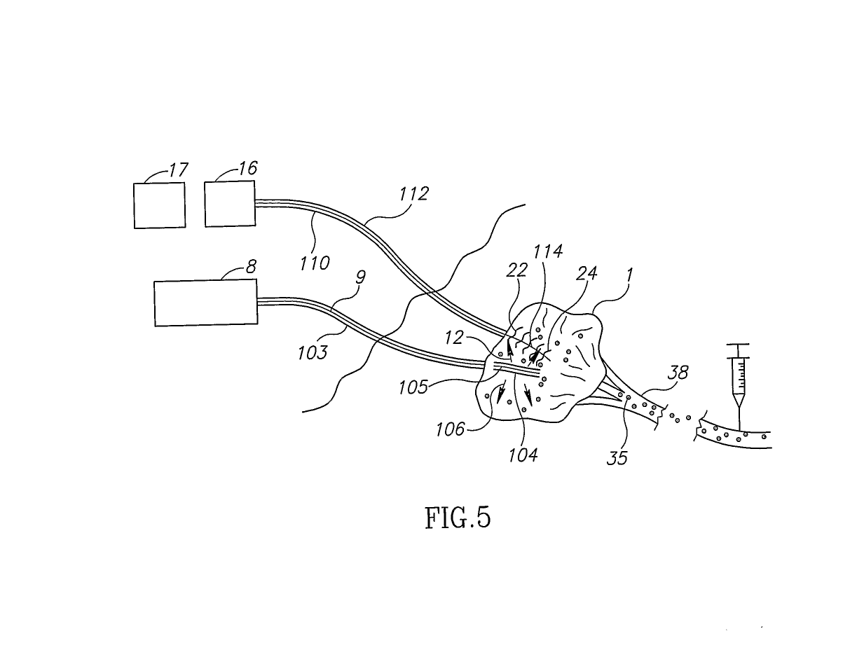

FIG. 5 illustrates a treatment of internal targeted tissue according to one currently preferred embodiment of the present invention. The target tissue 1 located deep within a subject body (hereinafter “deep tissue”) is administered with suitable nanoparticles 35 which are attached to the tissue cells mainly as clusters or retained mainly in the blood vessels, as agglomerates. An electromagnetic source 8, operable to generate electromagnetic pulse, is coupled and guided into a light guide 9 embedded in a catheter 103. An insertable tip protects the light guide 9 during insertion into the targeted tissue 1. The light guide 9 is coupled with a dispenser applicator 105, which is protected during insertion by an electromagnetic radiation transmissive tube 104. The dispenser applicator and its protective tube 104 are operable to dispense uniform radial electromagnetic radiation 12 so as to illuminate a significant portion of the target tissue 1 volume. An ultrasound source 16 driven by a driving means 17 is coupled to a metal wire 110 suitable for guiding ultrasound radiation. The metal wire 110 is isolated by a catheter 112 and comprises an exposed tip 114. The exposed metal tip 114 is operable to radially dispense the ultrasound radiation 22 into significant portion of the targeted tissue 1 volume. Each time the nanoparticle clusters are exposed to the electromagnetic radiation 106, they generate microbubble cloud 24. The microbubble cloud 24 absorbs a portion of the ultrasound radiation 22 power and convert it into heat, which is emitted to the tissue 1 volume. The electromagnetic source 8 may be repetitive to conserve the desired microbubble cloud 24 density within the targeted tissue 1 volume.

According to certain embodiments, the nanoparticles are induced to generate clusters or agglomerates. According to one embodiment, the stimulation to generate agglomerate is provided by the electromagnetic source or the ultrasound source. According to another embodiment the clustering is triggered by addition of complementary nanoparticles that interact with the nanoparticles attached to the tissue.

The systems and methods of the present invention have several important potential applications in the field of cancer treatment. For example, metastatic prostate cancer is a leading cause of mortality in American men. Estimates indicate that greater than one in every eleven men in the U.S. will develop prostate cancer. Localized prostate cancer is generally treated with either radical prostatectomy or radiation therapy. Both of these procedures are plagued by significant morbidity. Using the minimally invasive treatment of the invention can dramatically improve cancer therapy, including treating prostate cancer as well as breast cancer, brain cancer lung cancer etc.

Administration of a composition comprising nanoparticles to a cell, tissue or organism can follow general protocols for the administration of chemotherapeutics, taking into account the toxicity, if any. It is expected that the treatment cycles would be repeated as necessary. In particular embodiments, it is contemplated that various additional agents may be applied in any combination with the present invention.

Various combination regimens of inducing hyperthermia according to the teaching of the present invention in combination with one or more agents can be also employed.

Chemotherapeutic agents that can be used in combination with the present invention include, but are not limited to, 5-fluorouracil, bleomycin, busulfan, camptothecin, carboplatin, chlorambucil, cisplatin (CDDP), cyclophosphamide, dactinomycin, daunorubicin, doxorubicin, estrogen receptor binding agents, etoposide (VP 16), farnesyl-protein transferase inhibitors, gemcitabine, ifosfamide, mechlorethamine, melphalan, mitomycin, navelbine, nitrosurea, plicomycin, procarbazine, raloxifene, tamoxifen, taxol, temazolomide (an aqueous form of Dacarbazine), transplatinum, vinblastine and methotrexate, vincristine, or any analog or derivative variant of the foregoing. These agents or drugs are categorized by their mode of activity within a cell, for example, whether and at what stage they affect the cell cycle. Alternatively, an agent can be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, corticosteroid hormones, mitotic inhibitors, and nitrosoureas, hormone agents, miscellaneous agents, and any analog or derivative variant thereof.

Chemotherapeutic agents and methods of administration, dosages, etc. are well known to those of skill in the art (see for example, the “Physicians Desk Reference”, Goodman & Gilman's “The Pharmacological Basis of Therapeutics”, “Remington's Pharmaceutical Sciences”, and “The Merck Index, Eleventh Edition”, incorporated herein by reference in relevant parts). Some variation in dosage will necessarily occur depending on the condition of the subject being treated. The person responsible for administration will, in any event, determine the appropriate dose for the individual subject. Of course, all of these dosages and agents described herein are exemplary rather than limiting, and other doses or agents may be used by a skilled artisan for a specific patient or application. Any dosage in-between these points, or range derivable therein is also expected to be of use in the invention.

The system and/or methods of the present invention can be further utilized for joining tissue. The cell or tissue and the solder material to be joined are first administered with nanoparticles. The electromagnetic source is operated to generate microbubbles around the nanoparticles and the ultrasound is absorbed in the microbubbles cloud thus releasing heat to the cell or tissue and the soldering material.

According to certain embodiments, the method of joining tissue is used for procedures such as closure of skin wounds, vascular anastamosis, occular repair, nerve repair, cartilage repair, and liver repair. The method may be further used for joining tissue to non-tissue material.

According to one embodiment, at least a portion of the nanoparticles is mixed with one or more proteins. Specific embodiments of protein/nanoparticles systems include nanoparticles mixed with albumin, fibrinogen, collagen, elastin, fibronectin, laminin, chitosan, fibroblast growth factor, vascular endothelial cell growth factor, platelet-derived growth factor, epidermal growth factor, or insulin-like growth factor or any combination thereof. Alternatively, at least a portion of the nanoparticles are mixed with one or more polymers. Specific embodiments of polymer/nanoparticle systems include nanoparticles mixed with polyethylene, polyethylene glycol, polystyrene, polyethylene terephthalate, polymethyl methacrylate, or any combination thereof. In another embodiment, at least a portion of the nanoparticles is mixed with one or more polymers and one or more proteins. According to further embodiment, at least a portion of the nanoparticles is bound to a chemical moiety. According to one currently preferred embodiment, at least a portion of the nanoparticles is bound to an antibody.

In another embodiment of the invention, the method of joining tissue to non-tissue material comprises delivering nanoparticles to tissue and to non-tissue material, and exposing these nanoparticles to electromagnetic radiation which produce microbubbles, followed by application of ultrasound which in turn is absorbed by the microbubbles thus releasing heat. According to certain embodiments, the nanoparticles are mixed with protein, polymer or a combination thereof. According to one embodiment, the non-tissue is a medical device. In another embodiment, the non-tissues comprise engineered tissue.

The initial step of the present invention starts with the generation of microbubble cloud within the targeted volume. Next the microbubbles are exposed to continuous or pseudo continuous ultrasound radiation. However, the ultrasound radiation can be applied in short intense time period. In this way, collateral damage is minimized. Such an approach could be used to remove non-cellular non-tissue material, such as coronary plaque. The general methodology has additional uses in the area of cosmetic enhancements. Intense localized hyperthermia can be used to kill fat cells or to remove unsightly skin formations, among other potential cosmetic applications including, but not limited to, treating vascular lesions, pigmented lesions or acne; reducing wrinkles; and stretching marks.

Diagnostic Applications

Diagnostic imaging is an important tool for identification and 3-dimensional location of diseased tissue and cells. Diagnostic imaging can also indicate the location and boundaries of viable diseased cell or tissue during and after certain treatments. Diagnostic imaging can be further used for guided treatment, which is a common method to supervise minimally invasive treatment procedures. The most common diagnostic imaging modalities used for guided treatment are MRI, X-ray and ultrasound.

Ultrasound diagnostic imaging of diseased tissues is nowadays performed after administering contrast agents to the patient. When ultrasound waves encounter low-density high elasticity interfaces (like contrast agents), the changes in acoustic impedance result in a more intense reflection of sound waves and a more intense signal in the ultrasound image. These contrast agents size is a few microns and they are typically coated with attachment promoters which enhance their tendency to attach to the targeted tissue.

The present invention now discloses that microbubbles produced according to the teaching of the present invention are also useful as contrast agent, as they affect the ultrasound imaging of their immediate environment. Their main advantages over ordinary contrast agents include continuous renewable supply of microbubbles, localization of microbubbles within the suspected tissue and around the suspected cells, and filling the complete volume of the suspected tissue. These advantages enable continuous and convenient diagnostic imaging of additional tissue parameters like tissue temperature, tissue coagulation level and expansion of therapeutic materials within the tissue.

The microbubble size of the microbubbles produced according to the teaching of the present invention is suitable to efficiently interact with 1-3 MHz ultrasound, the typical range for ultrasound diagnostic imaging of a tissue loaded with contrast agents. The diagnostic imaging is typically conducted by the reflected sub- or second harmonic ultrasound wave which can easily be distinguished from strong fundamental harmonic reflections from bones and interfaces in the body. Imaging methods like pulse-to-pulse differentiation and Doppler techniques can be easily utilized for extracting additional tissue parameters distribution within the suspected tissue.

The ultrasonic imaging system for diagnosing a cell or a tissue preloaded with nanoparticles of the present invention comprises an electromagnetic radiation source configured to irradiate the nanoparticles to induce the production of microbubbles by said nanoparticles; an imaging ultrasonic wave generating source configured to irradiate the microbubbles as to enhance the ultrasound imaging contrast of said cell or tissue administered with said nanoparticles; driving means coupled to the imaging ultrasonic wave generating source for driving said imaging ultrasonic source with a drive signal to generate imaging ultrasonic waves; and an ultrasound probe.

The imaging can be conducted through separate diagnostic probe using ordinary techniques as are known in the art. For example short pulse train has a peak power which is in accordance with the FDA diagnostic ultrasound power regulations but sufficient for obtaining resolvable signal of the microbubbles at all the regions within the suspected tissue that are filled with microbubbles. Typically, the probe employs reflected sub- or second harmonic ultrasound wave which can easily be distinguished from strong fundamental harmonic reflections from bones and interfaces in the body. Various imaging techniques, like pulse sequencing (CPS) may be employed to obtain additional tissue parameters like temperature and coagulation level. The probe signal could be processed for scalar data or for obtaining 2-dimensional image using the B-mode processing method.

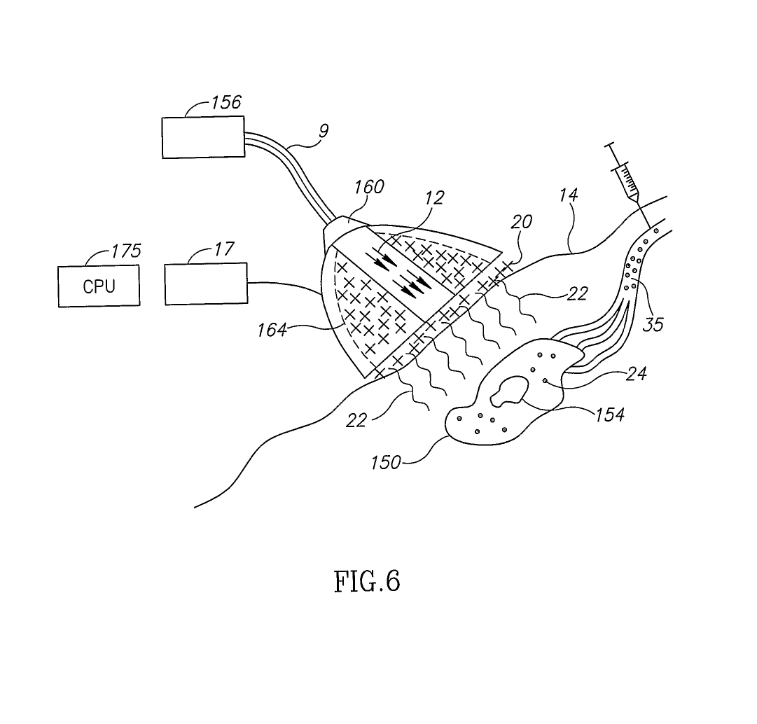

According to one currently preferred embodiment, a scheme of an apparatus for diagnostic imaging of a tissue or a cell suspected for a disease or disorder (hereinafter “a suspected cell/tissue”), wherein the cell/tissue is located near the outer surface of a subject body (hereinafter “a shallow tissue”) is illustrated in FIG. 6. The suspected shallow tissue 150 is administered with nanoparticles 35 designed to be attached to the suspected cells 154 mainly as clusters. A pulsed electromagnetic source 156 is coupled and guided into a light guide 9 and through a coupling unit 10 as to form a sufficiently wide and uniform electromagnetic beam 12 which illuminate the suspected tissue 150 through the patient accessible surface 14. A focusing ultrasound source/probe 164 driven by a driver/receiver 166 is located coaxially with the uniform beam 162 on an accessible patient surface 14 and uses suitable gel 20 to couple significant portion of the ultrasound radiation 22 to the suspected tissue 150 volume illuminated by the electromagnetic source 156. The ultrasound source/probe is connected through the driver/receiver 166 to a CPU 175, operable to process the ultrasound signals reflected from the examined tissue and process them to a displayable image.

The ultrasound attenuation level of the microbubble cloud can be excessive in some cases. At times, the imaging of the targeted tissue volume is desired instead of its boundary. In such case it is useful to reduce the microbubble density. In such case, the ultrasound radiation intensity is reduced, thus reducing the concentration of microbubbles maintained by the combined action of the ultrasound and the electromagnetic source. The cell or tissue is imaged by reception of the reflected ultrasound radiation in the probe 164.

The exposed nanoparticles clusters generate microbubbles cloud 24 upon exposure to the pulsed electromagnetic beam. The ultrasound radiation 22 stabilizes the microbubble cloud 24 to a level it can generate a contrast image of the suspected cells 154 in respect to surrounding healthy tissue. Thus, the suspected cells 154 can be imaged and detected by the ultrasound source/probe and CPU 175.

According to one embodiment, the electromagnetic source is operated in an intermittent and pulsed mode while the ultrasound probe emits continuous or pseudo continuous ultrasound radiation.

According to one embodiment, the electromagnetic source is a pulsed infrared light source. According to a currently preferred embodiment the ultrasound probe radiation frequency is between 0.5 and 7.5 MHz.

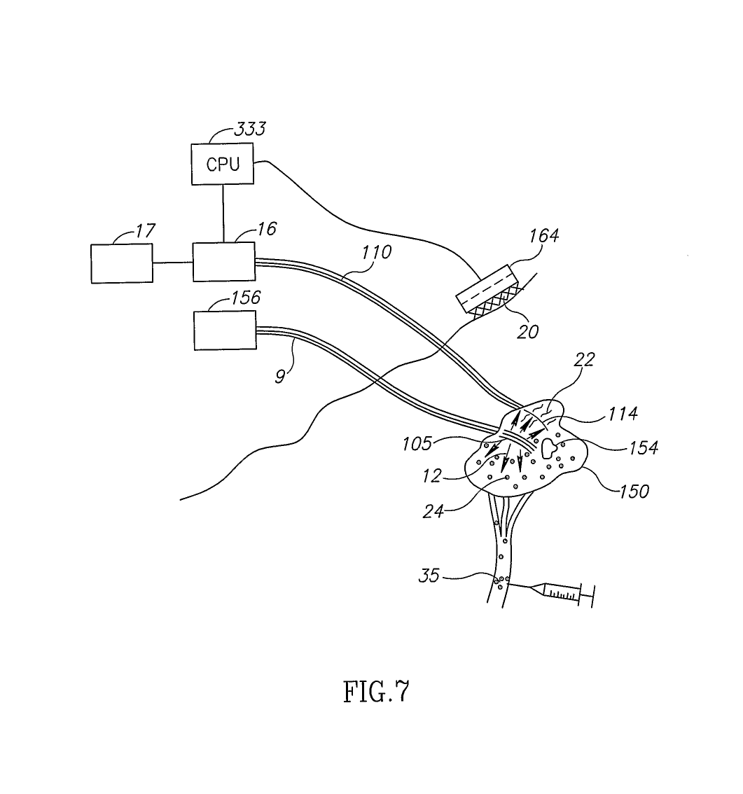

FIG. 7 illustrate a currently preferred scheme of an apparatus and method suitable for detection of suspected cells or tissue wherein the examined cell/tissue is located deep within a subject body (hereinafter “a deep cell/tissue”). The suspected tissue 150 is administered with suitable nanoparticles 35 which are designed to be target at and attached to the suspected cells 154 within the tissue, mainly as clusters. A pulsed electromagnetic source 156 is coupled to a suitable light guide 9 connected to an applicator tip 309 which disperse the electromagnetic radiation 106 mainly in radial direction. An ultrasound source 16 driven by a driving means 17 is coupled with a suitable catheter with a catheter operative to transport the ultrasound radiation with minimal losses to a metal wire 114 tip, inserted into the suspected tissue so as to couple large portion of its radiation 22 to the suspected tissue 150 volume illuminated by the applicator tip 309.

Exposure of the nanoparticle clusters on the suspected cells 154 to the electromagnetic radiation 106 generates microbubbles cloud 24 which is sustained by the action of the ultrasound radiation 22. A diagnostic ultrasound probe 164 located on an accessible patient surface with a suitable coupling gel 20 is operable to send ultrasound signals and receive the ultrasound signals reflected from the microbubble cloud 24 around the suspected cells 154. The received signals are in turn transferred from the probe 164 to a CPU 333 which processes them and generates an image consisting the suspected cells 154 thus identified within the tissue. The method for the detection of suspected cells in an examined deep tissue, is also useful for performing vascularized tissue imaging during HIFU therapy using reduced intensity pulsed ultrasound radiation. The suitable nanoparticles 35 are administered to the targeted tissue, attach to the suspected cells 154 as clusters, and in turn illuminated by the pulsed electromagnetic radiation 106. The generated microbubbles cloud is stabilized by the ultrasound radiation 22 such that it can be detected as to enable imaging of the suspected cells 154 by the ultrasound probe 164 and CPU 333.

According to one currently preferred embodiment, the electromagnetic source is operated in an intermittent and pulsed mode while the ultrasound source is operated continuously; the electromagnetic source is a pulsed infrared light source; and the ultrasound radiation frequency is between 0.1 and 7.5 MHz.

Real time imaging during therapeutic treatment becomes a widely accepted procedure for treatment of diseased tissue, especially for tissue located deep in the patient body. It is used to localize the administration of chemotherapy, widen the use of minimally invasive treatment and enable new and advanced treatments with minimal collateral damage, and hence, reduce the chance for complications.

Major advantages to real-time imaging during therapeutic treatment according to the present invention are: (1) real-time visualization of a treatment site is very reassuring to the medical therapist, in confirming that each of the energies are applied to the correct tissue and mutual position, especially in cases where the targeted tissue is located near a vital organ; (2) the treatment can be stopped when a therapeutic produced lesion has grown to the point at which it begins to extend beyond the desired treatment site; (3) the electromagnetic and ultrasound radiation sources positions may be readjusted in order to compensate for tissue movement within the patient's body due to breathing or for other reasons; (4) the combined imaging and therapeutic treatment can be accomplished much faster than in the past, when it was necessary to render treatment, stop the treatment, image the site, and then continue the treatment; (5) viewing the treatment site is very useful in cases where other modalities (e.g., chemotherapy) are employed during the treatment, in order to localize and minimize the chemotherapy dose.

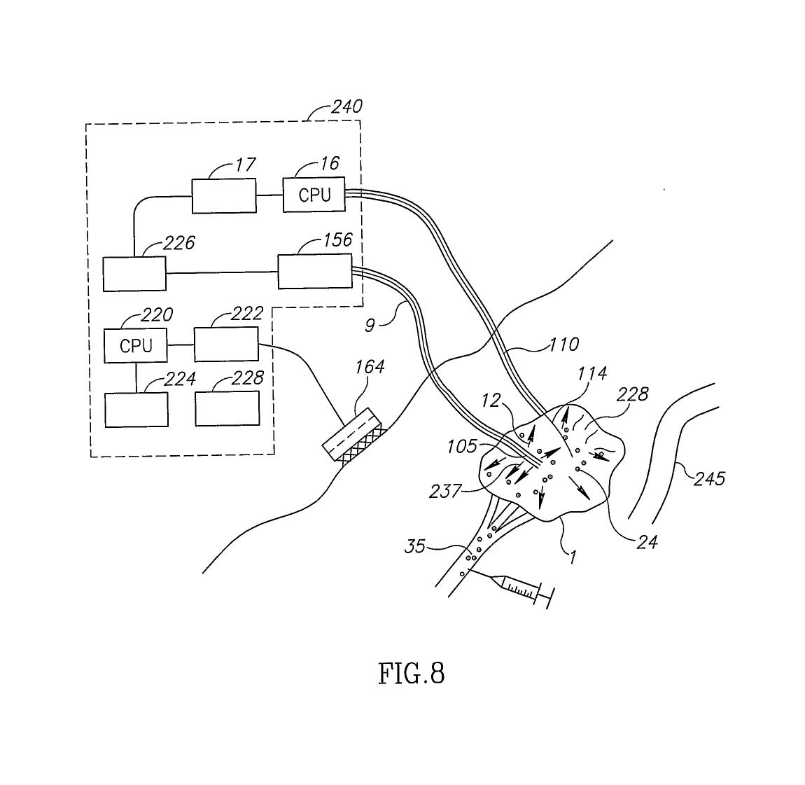

A preferred system 200 for guided treatment of a deep tissue is illustrated in FIG. 8. The system comprises: pulsed electromagnetic source 156, a suitable catheter with encased light guide 9 connected to a dispensing applicator 105; a suitable ultrasound driver 17 drives the ultrasound source 16 coupled with a metal wire 110 encased in a catheter and connected to the metal wire tip 114; an ultrasound probe 164 connected to a CPU 220 through a signal conditioning unit 222; a display unit 224 for displaying ultrasound images; a control panel 226 for controlling the treatment procedure, and a data bank 228. The deep-targeted tissue 1 located near a main blood vessel 245 is administered with suitable nanoparticles 35 which after certain period are attached to the tissue cells mainly as clusters and may be retained in the blood vessels or within the targeted tissue volume mainly as agglomerates. The electromagnetic source 156 sends electromagnetic radiation through a suitable light guide 9 to the dispensing applicator 105 operable for dispersing the electromagnetic radiation 106 mainly in radial direction to a predetermined volume within the targeted tissue 1 volume. An ultrasound source 16 transmits ultrasound radiation through the metal wire 110 operative to transport ultrasound radiation to a metal wire tip 114 inserted within the targeted tissue 1 so as to couple large portion of the ultrasound radiation to the targeted tissue 1 volume illuminated by the dispensing applicator 105.

Each time when the nanoparticles clusters or agglomerates within the targeted tissue 100 are exposed to the pulsed electromagnetic radiation 106, they generates microbubble cloud 24 which in turn is stabilized by the dispensed ultrasound radiation and converts a significant portion of the ultrasound radiation into heat 237 emitted to the targeted tissue 1.

An ultrasound probe 164 located on a selected location on the patient skin or internal cavities surface, is operable to send ultrasound signals and receive the ultrasound echo signals reflected through the targeted tissue. The echo signals received in the probe 164 are conditioned by a conditioning circuit 222 and routed to a CPU 220 which processes them and generates an image of the targeted tissue 1 during the treatment. The metal wire tip 114 cannot be used for 2-dimensional diagnostic imaging.

In a preferred embodiment of the guided treatment, the ultrasound radiation for microbubble stabilization and heating is delivered by an external ultrasound source coupled with a free patient body surface and coupled with a slightly focusing apparatus so as to irradiate the targeted tissue with relatively wide beam of low power ultrasound radiation.

Ultrasound guided treatment of deep targeted tissue according to the present invention involves the following steps (FIG. 8): Administering nanoparticles 35 to the patient body or the targeted tissue 1 and enable accumulation of nanoparticles within the targeted tissue 1 (step I); placing the ultrasound probe 164 on a suitable location on the free patient surface and coupling with suitable gel (step II); operating the ultrasound probe 164 in diagnostic imaging mode and use the image for safe insertion of catheter with metal wire 110 and a catheter with light guide 9 into the targeted tissue 1 (step III); turn on the electromagnetic source 156 and ultrasound source 16 and switch the ultrasound probe 164 to the suitable parameters of guided treatment mode (step IV); Continue the ultrasound treatment until the designated volume (or whole volume) of the targeted tissue 1 is exposed to temperature and time (typically a few minutes) sufficient for the desired treatment (step V); as necessary, the ultrasound catheter tip 114 and electromagnetic radiation catheters tip 105 are moved to new location, steps IV and step V are repeated until the whole volume of the targeted tissue 1 (or a plurality of targeted tissues) are exposed to temperature and time sufficient for the desired treatment.

Alternatively, the targeted cells or tissue may be imaged during the treatment using a photo-acoustic method (for example, as described in Niederhauser J. J., Real-Time Biomedical Optoacoustic Imaging, PhD Dissertation, Swiss Federal Institute of Technology Zurich 2004, incorporated herein in its entirety by reference}. The pulsed electromagnetic radiation source 156 (FIG. 8) is absorbed in the nanoparticles 35 which in turn rapidly heat the surrounding liquid. Since water and body liquids are almost incompressible, the isocoric heating generate a significant shock wave, mainly within the targeted tissue volume. The acoustic probe 164 attached to the patient skin receives the generated shock waves and uses signal conditioning unit 222 and CPU 220 to generate the suspected cells image.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-73. (canceled)

74. A system for localized delivery of heat to a cell or a tissue preloaded with nanoparticles comprising:
a. an electromagnetic radiation source configured to irradiate the nanoparticles to induce the production of microbubbles by said nanoparticles;
b. a therapeutic ultrasonic wave generating source configured to irradiate the microbubbles as to induce heat production by said microbubbles; and
c. driving means coupled to the therapeutic ultrasonic wave generating source for driving said therapeutic ultrasonic source with a drive signal to generate therapeutic ultrasonic waves.

75. The system according to claim 74, further comprising a light guide coupled to the electromagnetic radiation source to target the electromagnetic radiation to the cell or tissue.

76. The system according to claim 74, further comprising a focusing apparatus coupled to the therapeutic ultrasonic wave generating source.

77. The system according to claim 74, wherein the photothermal cross-section of the preloaded nanoparticles is enhanced to at least the physical cross-section of said nanoparticles.

78. The system according to claim 74, wherein the preloaded nanoparticles are present at a concentration in the range selected from the group consisting of 10⁵ to 10⁹ nanoparticles/cm³ and 3*10⁵ to 3*10⁷ nanoparticles/cm³.

79. The system according to claim 74, wherein the electromagnetic radiation source is selected from the group consisting of a plurality of light emitting diode (LED) lamp, gaseous flash lamp, diode laser pumped flash lamp, solid-state laser, diode laser, and a gaseous laser.

80. The system according to claim 74, wherein the electromagnetic radiation source provides radiation selected from the group consisting of ultraviolet radiation, visible radiation and infrared radiation.

81. The system according to claim 80, wherein the electromagnetic radiation is infrared radiation in the range of from about 800 nm to about 1300 nm.

82. The system according to claim 74, wherein the electromagnetic radiation source provides radiation in a repetitive pulse mode wherein the pulse width is in the range of from 0.01 μsec to 10 μsec.

83. The system according to claim 74, wherein the therapeutic ultrasonic source comprises a housing, wherein the housing comprises at least one piezoelectric transducer element made of a material selected from the group consisting of quartz, barium titanate, lead zirconium titanate and poly(vinylidene fluoride).

84. The system according to claim 74, wherein the therapeutic ultrasonic source provides ultrasound radiation selected from the group consisting of continuous wave mode, pulsed wave mode and modulated wave mode.

85. The system according to claim 84, wherein the pulsed wave mode has a pulse width in the range of from 1 microsecond to about 0.5 second and wherein the pulse is synchronized with the electromagnetic radiation pulse.

86. The system according to claim 74, wherein the therapeutic ultrasonic source provides ultrasound radiation in a frequency range of from about 0.5 MHz to about 7.5 MHz.

87. The system according to claim 74, wherein the therapeutic ultrasonic source provides ultrasound radiation in a peak power level in the range of from about 0.05 W/cm² to about 20 W/cm².

88. The system according to claim 74, wherein the therapeutic ultrasonic source provides ultrasound radiation in an average power level in the range of from about 0.125 W/cm² to about 3 W/cm².

89. The system according to claim 74, wherein the driving means comprises radio-frequency (RF) signal generator, and further comprises an amplifier that amplifies the RF signal to produce a drive signal.

90. A method for inducing localized delivery of heat to a cell or a tissue comprising:
a. administering a plurality of nanoparticles to the cell or tissue;
b. irradiating the nanoparticles administered to said cell or tissue by electromagnetic radiation, as to induce the production of microbubbles; and
c. exposing the microbubbles of step (b) to ultrasound radiation;
wherein said microbubbles emit heat upon exposure to the ultrasound radiation.

91. The method according to claim 90, wherein the nanoparticle concentration is in the range selected from the group consisting of 10⁵ to 10⁹ nanoparticles/cm³ and 3*10⁵ to 3*10⁷ nanoparticles/cm³.

92. The method according to claim 90, wherein the nanoparticles are selected from the group consisting of nanoparticles comprising a metal component selected from the group consisting of gold, silver, copper, platinum, palladium, lead, and iron and non-metallic nanoparticles.

93. The method according to claim 92, wherein the non-metallic nanoparticles are carbon nanoparticles.

94. The method according to claim 90, wherein photothermal cross-section of the nanoparticles is enhanced to at least the physical cross-section of said nanoparticles.

95. The method according to claim 90, wherein the nanoparticles are coated with a material which enhances said nanoparticle tendency to form clusters or agglomerates following exposure to an external stimulus selected from the group consisting of electromagnetic radiation, ultrasound radiation shock wave or any combination thereof.

96. The method according to claim 90, wherein the nanoparticles are coated with a material which prevents said nanoparticles from forming clusters, wherein the material is neutralized following exposure to an external stimulus.

97. The method according to claim 96, wherein the external stimulus is provided by administering complementary nanoparticles to the cell or tissue wherein the complementary nanoparticles are designed to neutralize the coating material.

98. The method according to claim 90, wherein the nanoparticles are coupled to at least one type of molecules, wherein the molecules specifically bind to the cell or tissue.

99. The method according to claim 98, wherein the binding is by formation of an antigen-antibody complex or by formation of a ligand-receptor complex.

100. The method according to claim 90, wherein the nanoparticle diameter is in the range of from about 10 to about 1,000 nanometer.

101. The method according to claim 90, wherein the nanoparticles have an external shape selected from the group consisting of spherical shape, cubic shape, oval shape and rod shape.

102. The method according to claim 90, wherein the nanoparticle structure is selected from the group consisting of solid structure, core/shell structure, hollow structure, tubular structure and star-like structure.

103. The method according to claim 90, wherein the electromagnetic radiation is selected from the group consisting of ultraviolet radiation, visible radiation and infrared radiation.

104. The method according to claim 103, wherein the infrared radiation is in the range of from about 800 nm to about 1300 nm.

105. The method according to claim 90, wherein the electromagnetic radiation is administered in a repetitive pulse mode, wherein the pulse width is in the range of from 0.01 μsec to 10 μsec.

106. The method according to claim 90, wherein the ultrasound radiation is applied in a mode selected from a continuous wave mode and a pulsed wave mode.

107. The method according to claim 106, wherein the pulse width is in the range of from 1 microsecond to about 0.5 second.

108. The method according to claim 90, wherein the ultrasound radiation frequency is in the range of from about 0.5 MHz to about 7.5 MHz.

109. The method according to claim 90, wherein the ultrasound radiation peak power level is in the range of from about 0.05 W/cm² to about 20 W/cm².

110. The method according to claim 90, wherein the ultrasound radiation average power level is in the range of from about 0.125 W/cm² to about 3 W/cm².

111. The method according to claim 90, wherein the electromagnetic radiation is applied through a light guide, wherein the light guide is located adjacent to the cell or tissue.

112. The method according to claim 90, wherein the microbubbles are exposed to the ultrasound radiation through an insertable applicator, wherein the tip of the applicator is located adjacent to the cell or tissue.

113. The method according to claim 90, further comprising the step of exposing the cell or tissue to electric field optimized to cause sensitization of said cell or tissue prior to nanoparticle irradiation with the electromagnetic radiation.

114. The method according to claim 90, for treating a tumor cell or tissue selected from the group consisting of malignant and non-malignant tumor cell or tissue.

115. The method according to claim 114, wherein the method is applied in combination with additional anti-tumor therapy.

116. The method according to claim 90, for dissolving a blood clot.

117. The method according to claim 90, for reducing the size of or removing at least one stone from a kidney.

118. The method according to claim 90, for treating inflammation in a cell or a tissue.

119. The method according to claim 90, for joining a tissue.

120. The method according to claim 119, wherein the tissue is joined to another tissue.

121. The method according to claim 119, wherein the tissue is joined to a non-tissue material.

122. The method according to claim 90, for a cosmetic treatment of targeted skin regions selected from the group consisting of treating vascular lesions, pigmented lesions, acne and unsightly skin formation; removing unwanted hair; and reducing stretch marks or wrinkles.

123. An ultrasonic imaging system for diagnosing a cell or a tissue preloaded with nanoparticles comprising:
a. an electromagnetic radiation source configured to irradiate the nanoparticles to induce the production of microbubbles by said nanoparticles;
b. at least one imaging ultrasonic wave generating source configured to irradiate the microbubbles as to enhance the ultrasound imaging contrast of said cell or tissue administered with said nanoparticles; an ultrasound probe;
c. driving means coupled to the imaging ultrasonic wave generating source for driving said imaging ultrasonic source with a drive signal to generate imaging ultrasonic waves; and
d. an ultrasound probe.

124. The system according to claim 123, further comprising a light guide coupled to the electromagnetic radiation source to target the electromagnetic radiation to the cell or tissue.

125. The system according to claim 123, further comprising a focusing apparatus coupled to the imaging ultrasonic source.

126. The system according to claim 123, wherein the photothermal cross-section of the preloaded nanoparticles is enhanced to at least the physical cross-section of said nanoparticles.

127. The system according to claim 123, wherein the preloaded nanoparticles are present in a concentration in the range selected from the group consisting of 10⁵ to 10⁹ nanoparticles/cm³ and 3*10⁵ to 3*10⁷ nanoparticles/cm³.

128. The system according to claim 123, wherein the electromagnetic radiation source provides radiation selected from the group consisting of visible radiation and infrared radiation.

129. The system according to claim 128, wherein the electromagnetic radiation is infrared radiation in the range of from about 800 nm to about 1300 nm.

130. The system according to claim 123, wherein the electromagnetic radiation source provides radiation in a repetitive pulse mode, wherein the pulse width is in the range of from 0.01 μsec to 10 μsec.

131. The system according to claim 123, wherein the imaging ultrasonic source emission mode is selected from the group consisting of short pulse trains and Contrast Pulse Sequencing (CPS).

132. The system according to claim 123, wherein the driving means and the imaging ultrasonic source are configured for two-dimensional ultrasound imaging in a B-mode.

133. A method for ultrasonic imaging of a cell or a tissue, comprising:
a. administering nanoparticles to the cell or tissue;
b. irradiating the nanoparticles administered to said cell or tissue by electromagnetic radiation, as to induce the production of microbubbles; and
c. exposing the microbubbles of step (b) to ultrasound radiation;
wherein said microbubbles enhance the ultrasound imaging contrast of said cell or tissue administered with said nanoparticles.

134. The method according to claim 133 for diagnosing a diseased cell or tissue surrounded by healthy cells or tissue.

135. The method according to claim 133 for imaging a cell or tissue during a therapeutic treatment.
